(19) United States
(12) Patent Application Publication
(10) Pub. No.: US 2009/0214439 A1
(43) Pub. Date: Aug. 27, 2009

(54) Fluorinated Carbohydrates and Their Use in Tumor Visualization, Tissue Engineering, and Cancer Chemotherapy

(75) Inventors: Krishna Kumar, Cambridge, MA (US); Marc D'Alarcao, San Jose, CA (US); Laila Dafik, Somerville, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Trustees of Tufts College, Boston, MA (US)

(21) Appl. No.: 12/293,043

(22) PCT Filed: Mar. 15, 2007
(86) PCT No.: PCT/US07/64060

§ 371 Date: Feb. 24, 2009

Related U.S. Application Data
(60) Provisional application No. 60/782,487, filed on Mar. 15, 2006.

(51) Int. Cl. A61K 49/10; C07H 5/06; C12N 5/00; A61K 31/7008; A61P 35/00

(52) U.S. Cl. 424/9.35; 536/29.1; 435/325; 514/42

(57) ABSTRACT

The present invention relates to fluorine-containing monosaccharides that are useful for forming extracellular fluorinated glycoconjugates. Methods of forming extracellular fluorinated glycoconjugates comprise the steps of contacting a cell with a fluorine-containing monosaccharide, and incubating the cell under conditions whereby the cell internalizes the fluorine-containing monosaccharide, or a derivative thereof, on the surface of the cell. The present invention also relates to the use of a fluorine-containing monosaccharide in cellular imaging using fluorine NMR. The invention additionally relates to the use of fluorine containing monosaccharides in the treatment of cancer and inflammatory disease.

### BACKGROUND OF THE INVENTION

Molecules decorating cell surfaces govern their gross physical properties, and also serve as biological mediators of interaction, recognition and function. Methods to graft systematically unnatural molecules onto cell surfaces have recently emerged, and provide powerful control over functionalities displayed. [Luchansky, S. J.; Goon, S.; Bertozzi, C. R. “Expanding the diversity of unnatural cell-surface sialic acids” ChemBioChem 2004, 5, 371-4; Kiick, K. L.; Saxon, E.; Tirrell, D. A.; Bertozzi, C. R. “Incorporation of azides into recombinant proteins for chemoselective modification by the Staudinger ligation” Proc. Natl. Acad. Sci. U.S.A. 2002, 99, 19-24; and Oetke, C.; Brossmer, R.; Mantey, L. R.; Hinderlich, S.; Isecke, R.; Reutter, W.; Keppler, O. T.; Pawlita, M. “Versatile biosynthetic engineering of sialic acid in living cells using synthetic sialic acid analogues” J. Biol. Chem. 2002, 277, 6688-95.] Such advances proffer the introduction of functionality and chemical reactivity not found in nature. The latter has been elegantly demonstrated in several instances where orthogonal functionalities, such as ketones and azides, have been displayed on the cell surface. [Saxon, E.; Bertozzi, C. R. “Cell surface engineering by a modified Staudinger reaction” Science 2000, 287, 2007-10; Yarema, K. J.; Mahal, L. K.; Bruehl, R. E.; Rodriguez, E. C.; Bertozzi, C. R. “Metabolic delivery of ketone groups to sialic acid residues—Application to cell surface glycoform engineering” J. Biol. Chem. 1998, 273, 31168-79; and Lemieux, G. A.; Bertozzi, C. R. “Chemoselective ligation reactions with proteins, oligosaccharides and cells” Trends Biotechnol. 1998, 16, 506-13.] Indeed, this strategy has been extended to modifying cell surfaces in living animals. [Kayser, H.; Zeitler, R.; Kannicht, C.; Grunow, D.; Nuck, R.; Reutter, W. “Biosynthesis of a Nonphysiological Sialic-Acid in Different Rat Organs, Using N-Propanoyl-D-Hexosamines as Precursors” J. Biol. Chem. 1992, 267, 16934-8; and Prescher, J. A.; Dube, D. H.; Bertozzi, C. R. “Chemical remodelling of cell surfaces in living animals” Nature 2004, 430, 873-7.] In most of these cases, the ultimate surface attachment has resulted from covalent reaction of a second exogenous reagent with bio-orthogonal reactivity, such as an O-caboxymethyl arylphosphine for Staudinger ligation with the initially expressed azide or an aminooxy compound or hydrazide for oxime and hydrazone formation respectively with a cell-surface expressed ketone.

Immiscibility of fluorocarbons with water and hydrocarbons. Interestingly, decorating cells with fluoroalkyl groups provides a non-covalently interacting surface that is both hydrophobic and lipophobic. The insolubility of fluorinated materials in both aqueous and organic media is easily rationalized. In aqueous solvent, the hydrophobic effect is sufficient to account for immiscibility. In nonpolar organic solvents, Hildebrand and Scott's theory of the solubility of non-electrolytes can be invoked. [Scott, R. L. “The Solubility of Fluorocarbons” J. Am. Chem. Soc. 1948, 70, 4090-3.]

Δ̅F̅₁̅=RT ln x₁+v₁(δ₁−δ₂)²φ₂² (1a)

Δ̅F̅₂̅=RT ln x₂+v₂(δ₁−δ₂)²φ₁² (1b)

On a simple level, the solubility parameter δ determines the extent to which nonpolar liquids are miscible. The partial molal free energy of a component in a mixture of two nonpolar liquids is given by a sum of the entropy of mixing and the heat of mixing where x₁ and x₂ are the respective mole fractions, v₁ and v₂ are the molal volumes, φ₁ and φ₂ are the volume fractions and δ₁ and δ₂ are the solubility parameters. δ is given by (ΔE^{v}/v)^{1/2}, where ΔE^{v} is the energy of vaporization of the pure component and v its molal volume at temperature T. When δ₁=δ₂, there is no heat of mixing and the two liquids form an ideal solution. When the differences in δ become substantial, phase separation occurs. Small inequalities in δ result in partial miscibility. Fluorous liquids are characterized by small values of δ, signifying exceedingly low propensities for intermolecular interactions. Thus, these liquids form an independent phase in most organic solvents and in water.

Therefore, by using fluoroalkyl modification, the inertness and bio-orthogonal non-covalent binding properties of these groups may be exploited. This strategy has been successfully employed in peptide and protein design studies and is emerging as a powerful tool to control non-covalent associations in biological systems. [Bilgicer, B.; Kumar, K. “De novo design of defined helical bundles in membrane environments” Proc. Natl. Acad. Sci. U.S.A. 2004, 101, 15324-9; Bilgicer, B.; Fichera, A.; Kumar, K. “A coiled coil with a fluorous core” J. Am. Chem. Soc. 2001, 123, 4393-9; Marsh, E. N. G. “Towards the nonstick egg: designing fluorous proteins” Chem. Biol. 2000, 7, R153-R7.; Tang, Y.; Ghirlanda, G.; Vaidehi, N.; Kua, J.; Mainz, D. T.; Goddard, W. A.; DeGrado, W. F.; Tirrell, D. A. “Stabilization of coiled-coil peptide domains by introduction of trifluoroleucine” Biochemistry 2001, 40, 2790-6; Tang, Y.; Ghirlanda, G.; Petka, W. A.; Nakajima, T.; DeGrado, W. F.; Tirrell, D. A. “Fluorinated coiled-coil proteins prepared in vivo display enhanced thermal and chemical stability” Angew. Chem., Int. Ed. 2001, 40, 1494-1496; Tang, Y.; Tirrell, D. A. “Biosynthesis of a highly stable coiled-coil protein containing hexafluoroleucine in an engineered bacterial host” J. Am. Chem. Soc. 2001, 123, 11089-90; and Niemz, A.; Tirrell, D. A. “Self-association and membrane-binding behavior of melittins containing trifluoroleucine” J. Am. Chem. Soc. 2001, 123, 7407-13.]

Biosynthesis and cell-surface expression of sialic acid-containing glycans. A number of cell surface lipids (e.g., gangliosides), as well as most cell surface proteins and many secreted proteins, incorporate sialic acid-containing glycans. These appended oligosaccharides are believed to be extensively involved in cell-cell recognition, communication, adhesion, and secreted protein lifetimes. Glycoproteins fall into two categories, N-linked or O-linked, depending on whether the glycan is attached to the γ nitrogen atom of an asparagine residue or the β oxygen atom of a serine or threonine residue.

While the structure of the protein portion of glycoproteins is genetically encoded, the glycosylation pattern is governed by co- and posttranslational glycosyltransferase enzymes in the endoplasmic reticulum and the Golgi apparatus. However, these enzymes are often present in limited amounts, so not all proteins passing through these organelles are glycosylated to the full extent possible. This leads to a significant degree of microheterogeneity in glycoprotein structure.

The sialic acid residues on glycoproteins are particularly important recognition elements because sialic acid is the only commonly incorporated monosaccharide to bear a negative charge without further modification. Thus, cells decorated with differentially sialylated proteins and lipids present drastically different sites for non-covalent interaction; indeed cells differing in this way often exhibit different phenotypes. This fact is dramatically illustrated by cells found in many metastatic tumors which are differently sialylated than those in untransformed cells in the same tissue. [Alper, J. “Glycobiology—Turning sweet on cancer” Science 2003, 301, 159-60.]

The biosynthesis of sialic acid-containing glycoproteins and glycolipids always includes a sialylation step, in which the sialic acid (N-acetylneuraminic acid; Neu5Ac; Sia) residue is transferred from CMP-Neu5Ac to a pendant saccharide attached earlier to the protein or lipid (see FIG. 1). This process is catalyzed by a sialyltransferase, of which eighteen mammalian types have been cloned. These fall into four general categories, depending on the linkage synthesized: Siaα2-6Gal, Siaα2-3Gal, Siaα2-8Sia, and Siaα2-6GalNAc. [Angata, T.; Varki, A. “Chemical diversity in the sialic acids and related alpha-keto acids: An evolutionary perspective” Chem. Rev. 2002, 102, 439-69.] While the known sialyltransferases have strict substrate specificities for the glycosyl acceptor, they are much more tolerant of structural diversity in the donor.

The CMP-Neu5Ac substrate for the sialyltransferases is biosynthesized in vertebrates from glucose, via the common intermediate UDP-GlcNAc (FIG. 1). This intermediate is converted to ManNAc-6-phosphate by the bifunctional enzyme UDP-GlcNAc 2-epimerase/ManNAc kinase. The sialic acid skeleton is generated by the addition of the three carbons of phosphoenolpyruvate to ManNAc-6-phosphate by Neu5Ac-9-phosphate synthetase. Removal of the phosphate group via Neu5Ac-9-phosphate phosphatase and transfer of a CMP group from CTP by CMP-Neu5Ac synthetase completes the process. The last step occurs in the nucleus of the cell and therefore requires a transporter to assist in the relocation of the CMP-Neu5Ac to the Golgi apparatus where it functions as a substrate of the sialyltransferase enzymes. Incorporation of unnatural sialic acid analogues into glycoproteins and glycolipids. In 1992, Werner Reutter's laboratory demonstrated that both N-propanoyl D-glucosamine and N-propanoyl D-mannosamine are converted in vivo (rat) to the corresponding N-propanoyl sialic acid and that this modified and unnatural sialic acid is subsequently displayed on the cell surface glycoproteins. [Kayser, H.; Zeitler, R.; Kannicht, C.; Grunow, D.; Nuck, R.; Reutter, W. “Biosynthesis of a Nonphysiological Sialic-Acid in Different Rat Organs, Using N-Propanoyl-D-Hexosamines as Precursors” J. Biol. Chem. 1992, 267, 16934-8.] Since this discovery, several groups have been utilizing the apparently promiscuous nature of the biosynthetic pathway to induce cells to display modified glycoproteins and glycolipids.

Bertozzi has exploited this strategy to display modified sialic acids that contain functional groups that are chemically reactive, but with bioorthogonal reactivity, on cell surface molecules. For example, her laboratory has demonstrated that N-levulinoylmannsosamine and N²-azidoacetylmannsosamine are each converted in vivo into the correspondingly modified sialic acid and displayed on the cell surface. [Yarema, K. J.; Mahal, L. K.; Bruehl, R. E.; Rodriguez, E. C.; Bertozzi, C. R. “Metabolic delivery of ketone groups to sialic acid residues—Application to cell surface glycoform engineering” J. Biol. Chem. 1998, 273, 31168-79; and Lemieux, G. A.; Bertozzi, C. R. “Chemoselective ligation reactions with proteins, oligosaccharides and cells” Trends Biotechnol. 1998, 16, 506-13.] The ketone group in the former and the azide group in the latter can then be derivatized by formation of a hydrazone or by Staudinger ligation, respectively. In both cases nothing else on the cell is reactive, so this provides a method to label selectively cell surface glycoconjugates in a covalent manner.

A similar strategy has been used to display unnatural polysialic acids on the surface of cells [Samuel, J.; Bertozzi, C. R. c “Chemical Tools for the study of polysialic acid” Trends in Glycoscience and Glycotechnology 2004, 16, 305-318].

The substrate specificity of the sialylic acid biosynthetic machinery has been explored. [Jacobs, C. L.; Goon, S.; Yarema, K. J.; Hinderlich, S.; Hang, H. C.; Chai, D. H.; Bertozzi, C. R. “Substrate specificity of the sialic acid biosynthetic pathway” Biochemistry 2001, 40, 12864-74.] The results indicate that the mannosamines bearing acyl groups with five or fewer linear carbons are effectively expressed on the cell surface via this pathway, but longer chains or branched chains drastically reduce the expression. By analyzing the buildup of intermediates, it was determined that the least promiscuous enzyme in the pathway is the ManNAc-6-kinase.

This realization lead to a modified strategy for incorporating unnatural sialic acid analogues into cell-surface molecules whereby the modified sialic acid rather than the modified mannosamine is added to the cell. Using this strategy a much more diverse set of modifications is tolerated, since the most selective enzyme in the pathway has been bypassed. By this method, sialylated glycoconjugates bearing N-acyl groups of up to seven linear carbons or even an aryl azide can be expressed on cell surfaces.

A major limitation in the current technology for expressing modified sialic acid residues on cell surfaces is that there is almost no information regarding the distribution of the modified residues among all of the sialylated glycans on the cell. It is likely that little or no selectivity exists, so N-linked and O-linked glycoproteins as well as glycolipids are presumed to be modified to similar extents. The heterogeneous display of modified sialic acid residues complicates careful, molecular-level studies of the mechanisms of any phenotypes exhibited by the modified cells. A simple method to characterize the distribution of modified sialic acids among cell surface molecules, and/or to modify specifically individual molecules or classes of molecules on the glycocalyx is needed.

Cell adhesion in disease processes. The adhesion of cells to each other and to the molecules of the extracellular matrix (ECM), such as fibronectin or collagen, is essential for proper tissue organization and function. Accordingly, numerous disease processes have been associated with changes in the adhesive properties of cells, including tumor metastasis, cardiovascular disease, and various other inflammatory diseases, such as multiple sclerosis, inflammatory bowel disease, Behcet's disease, arthritis, pelvic inflammatory disease, chronic obstructive pulmonary disease, asthma, inflammatory diseases of the thyroid, diabetes mellitus, lupus erythematosus; Kawasaki disease, immune thrombocytopenic purpura, necrotizing enterocolitis, nephritis, atherosclerosis, psoriasis, gout, and sarcoidisis. [Ezzat, S.; Asa, S. L. “The molecular pathogenetic role of cell adhesion in endocrine neoplasia” J. Clin. Path. 2005, 58, 1121-5; Murakami, Y. “Involvement of a cell adhesion molecule, TSLC1/IGSF4, in human oncogenesis” Cancer Sci. 2005, 96, 543-52; Swart, G. W. M.; Lunter, P. C.; van Kilsdonk, J. W. J.; van Kempen, L. “Activated leukocyte cell adhesion molecule (ALCAM/CD166): Signaling at the divide of melanoma cell clustering and cell migration?” Cancer Metastasis Rev. 2005, 24, 223-36; Nair, K. S.; Naidoo, R.; Chetty, R. “Expression of cell adhesion molecules in oesophageal carcinoma and its prognostic value” J. Clin. Path. 2005, 58, 343-51; Gassmann, P.; Enns, A.; Haier, J. “Role of tumor cell adhesion and migration in organ-specific metastasis formation” Onkologie 2004, 27, 577-82; King, J. A.; Ofori-Acquah, S. F.; Stevens, T.; Al-Mehdi, A. B.; Fodstad, O.; Jiang, W. G. “Activated leukocyte cell adhesion molecule in breast cancer: prognostic indicator” Breast Cancer Res. 2004, 6, R478-R87; Geng, J. G.; Chen, M.; Chou, K. C. “P-selectin cell adhesion molecule in inflammation, thrombosis, cancer growth and metastasis” Curr. Med. Chem. 2004, 11, 2153-60; Brauer, P. R. “MMPs—Role in cardiovascular development and disease” Front. Biosci. 2006, 11, 447-78; Kanwar, J. R. “Anti-inflammatory immunotherapy for multiple sclerosis/experimental autoimmune encephalomyelitis (EAE) disease” Curr. Med. Chem. 2005, 12, 2947-62; Danese, S.; Semeraro, S.; Marini, M.; Roberto, I.; Armuzzi, A.; Papa, A.; Gasbarrini, A. “Adhesion molecules in inflammatory bowel disease: Therapeutic implications for gut inflammation” Dig. Liver Dis. 2005, 37, 811-8; Al-Mutawa, S. A.; Hegab, S. M. “Behcet's disease” Clin. Exp. Med. 2004, 4, 103-31; and Szekanecz, Z.; Koch, A. E. “Therapeutic inhibition of leukocyte recruitment in inflammatory diseases” Curr. Opin. Pharm. 2004, 4, 423-8.]

Some cell types (for example, phagocytotic leukocytes, such as neutrophils or macrophages) must undergo stimulated changes in their adhesive properties in order to escape the bloodstream and migrate to the site of pathogen assault, which is often buried deep in tissues. This process of leukocyte tethering, rolling, activation, arrest, and ultimately extravasation by passage between vascular endothelial cells and the underlying basement membrane is mediated by a complex series of interactions between cell-surface glycoproteins on both leukocytes and endothelial cells. Tethering and rolling are thought to be mediated by interaction of selectins with sialylated glycoproteins, while arrest and extravasation are believed to be primarily mediated by interaction of integrins with ECM components as well as inflammation-induced ICAM-1 or VCAM-1 molecules. [Luster, A. D.; Alon, R.; von Andrian, U. H. “Immune cell migration in inflammation: present and future therapeutic targets” Nat. Immunol. 2005, 6, 1182-90.] The process is complicated by the fact that these adhesion molecules are involved in dynamic and interdependent signaling. For example, integrin participates in “inside-out” signaling that regulates its affinity for its extracellular binding partners as well as “outside-in” signaling that controls the cell's response to adhesion. [Ginsberg, M. H.; Partridge, A.; Shattil, S. J. “Integrin regulation” Curr. Opin. Cell Biol. 2005, 17, 509-16; and Kinashi, T. “Intracellular signalling controlling integrin activation in lymphocytes” Nat. Rev. Immunol. 2005, 5, 546-59.]

Because of the centrality of cell adhesion to leukocyte function and thus to inflammation, a large effort has been mounted to develop molecules that inhibit steps in the process as possible therapies for inflammatory disease. Furthermore, it is likely that tumor metastasis is at least partly due to the acquisition by tumor cells of leukocyte-like adhesion glycoproteins, enabling the cells to invade other tissues. [Kannagi, R.; Izawa, M.; Koike, T.; Miyazaki, K.; Kimura, N. “Carbohydrate-mediated cell adhesion in cancer metastasis and angiogenesis” Cancer Sci. 2004, 95, 377-84.] Therefore, molecules that inhibit adhesion may also find application in cancer chemotherapy.

### SUMMARY OF THE INVENTION

One aspect of the invention relates to fluorine-containing monosaccharides. Another aspect of the invention relates to oligosaccharides and polysaccharides comprising at least one fluorine-containing monosaccharide. In certain embodiments, said fluorine-containing monosaccharides are part of the sialic acid biosynthetic pathway. In certain embodiments, said fluorine-containing monosaccharides are mannosamines or sialic acids.

Another aspect of the invention provides for methods for forming extracellular fluorinated glycoconjugates comprising the steps of contacting a cell with a fluorine-containing monosaccharide; and incubating said cell under conditions whereby the cell internalizes said fluorine-containing monosaccharide and extracellularly expresses a glycoconjugate comprising said fluorine-containing monosaccharide, or a derivative thereof, on the surface of said cell. In certain embodiments, said extracellular fluorinated membrane-bound glycoconjugate reduces cellular adhesion. Another aspect of the invention relates to the use of the inventive compounds in cellular imagining using fluorine MRI. Yet another aspect of the invention relates to the use of the invention compounds in the treatment of cancer (e.g., tumor metastasis) and inflammatory diseases.

### BRIEF DESCRIPTION OF THE FIGURES

FIG. 1 depicts the biosynthesis of sialic acid-containing glycoproteins and glycolipids. The first three steps yielding the corresponding sialic acid are carried out in the cytoplasm; the sialic acid is then activated in the nucleus to the CMP form and transported to the Golgi where sialyltransferases attach it to proteins and lipids.

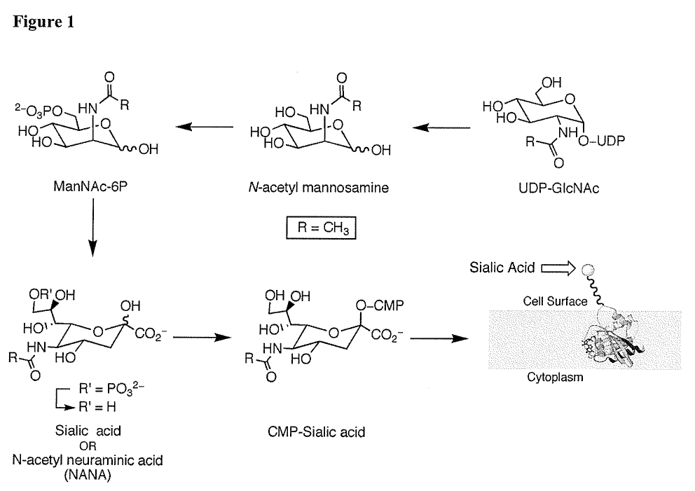

FIG. 2 depicts the synthesis of substituted mannosamines and neuraminic acids. The tetraacetylated compounds 4 and the peracetylated methyl ester of the corresponding N-acyl neuraminic acids 5 were used in feeding experiments. Conditions: (a) HBTU, DIEA/DMF, RCO₂H, 0° C.→rt; (b) Aldolase, Na-pyruvate, 37° C., 3 d; (c) Ac₂O, Pyridine (0.2 M), rt, 16 h; (d) AcCl, CH₃OH, rt, 12 h. Yields reported in parentheses are over two steps [(a) and (b)] for 4 and over three steps [(b), (d), (c)] for 5 starting from 2. Compounds with underlined numbers have not yet been prepared.

FIG. 3 depicts the conversion of exogenously added derivatives of mannosamine to corresponding sialic acids. Cytosolic fraction of Jurkat cells analyzed by LC ESI-MS after 36 hrs. of incubation with a) 4b and b) 4d. The ordinate represents relative intensity of the total ion current.

FIG. 4 depicts a visualization scheme for unnatural cell-surface sialic acids. Cells incubated with unnatural sialic acid derivatives are subjected to hydrolysis and then labeled with DMB. Comparison of an authentic standard labeled in an identical manner establishes the retention time on HPLC using a fluorescence detector. Integration of the area under the peak provides quantitation of the relative amounts of different sialic acids. The unnatural functionality is part of the R group on the C5 amino functionality. HPLC trace shown is for N-acetyl neuraminic acid (R═CH₃).

FIG. 5 depicts the structures of compounds tested for incorporation on cell surfaces. Compounds 4e, 4g, 5e, 5g, and 51_a were escorted and appended to cellular surfaces in the form of sialoglycoconjugates. The other compounds were either only minimally incorporated or not at all. The incorporation was not a function of ability to diffuse across the plasma membrane as all compounds were found in cytosolic fractions when not incorporated. Note that compounds found in the cytosol and expressed on the cell surface are in the free form (in the tetrahydroxyl form for compounds 4 and in the pentahydroxyl carboxylate form for 5).

FIG. 6 depicts the results of incubation of cells with 51 (Jurkat or HL60): minimal incorporation (<3% of total sialic acid). However, in both cases, examination of the cytosolic fraction revealed the presence of 51. The membrane passage of the unnatural sialic acid is not compromised.

FIG. 7 depicts the expression of compounds 5e and 5g on Jurkat, HL60 and HeLa cell surfaces. Fluorescence based detection of DMB conjugates was achieved by subjecting the membrane fraction to hydrolysis and labeling. The peak on the left is Neu5Ac and on the right is the unnatural fluorinated sialic acids. (a) Compound 5e is efficiently incorporated in Jurkat cells; (b) LC ESI-MS confirms mass and identity of 5e; (c) Cell surface expression of 5g by incubation of 4g with HL60 (but not Jurkat) (d) in HeLa cells. Uncolored peaks are unknown degradation products of DMB alone. NOTE: All material isolated from the cell surfaces is in the pentahydroxyl carboxylate form.

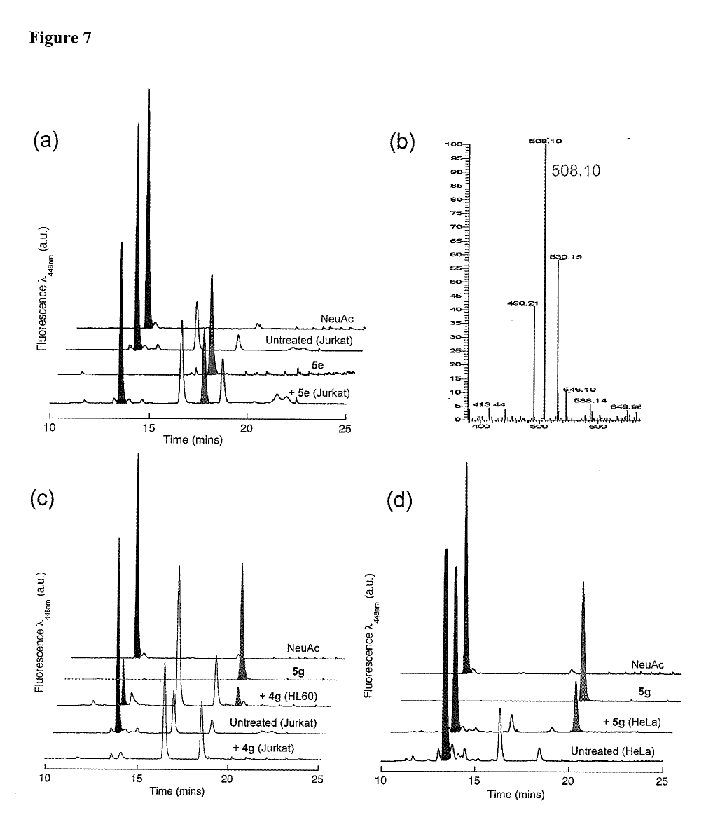

FIG. 8 depicts graphs showing that treatment of HL60-I deficient in UDP-GlcNAc 2-epimerase activity treated with 5g shows that roughly ˜75% of cell surface sialylation comes from exogenously added 5g. The sialic acid composition [Neu5Ac (left); 5g (right)] was visualized using DMB labeling and fluorescence HPLC detection. The other peaks are attributed to decomposition to excess DMB.

FIG. 9 depicts (a) adhesion of HL60 cells to plates coated with fibronectin in the presence of thapsigargin (Tg). The cells were incubated with compounds for 72 hours, washed with PBS and then allowed to adhere to plates. After 1 hr, unattached cells were removed by rinsing with PBS and then the number of cells quantified by addition of calcein-AM and fluorescence counting. (b) Similar assay as in (a) but with compounds 4a and 4d (data in (b) is taken from Horstkorte, R.; Rau, K.; Laabs, S.; Danker, K.; Reutter, W. “Biochemical engineering of the N-acyl side chain of sialic acid leads to increased calcium influx from intracellular compartments and promotes differentiation of HL60 cells” FEBS Lett. 2004, 571, 99-102).

FIG. 10 depicts adhesion of HL60 cells to plates coated with [a] E-selectin (Human CD62E) and [b] P-selectin (Human CD62P). The cells were incubated compound 5e and compound 5g for 72 hours, washed with PBS and then allowed to adhere to plates. After 1 hr, unattached cells were removed by rinsing with PBS and then the number of cells quantified by addition of calcein-AM and fluorescence counting.

FIG. 11 depicts selected modified sialic acids of the invention.

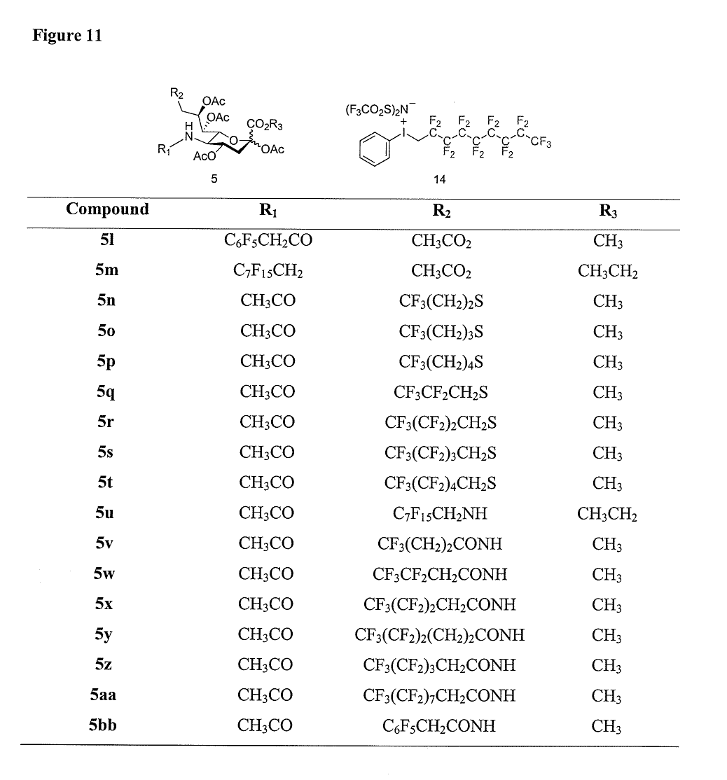

FIG. 12 depicts a proposed syntheses of fluorinated sialic acid analogues. a. Dowex H⁺, MeOH; b. TsCl, pyr; c. AcSK, DMF; d. NaOMe, MeOH; e. NaN₃, DMF; f. SnCl₂, MeOH; g. NaOEt, EtOH; h. R_{f}CH₂COCl, pyr or 14; i. Ac₂O, pyr; j. R_{f}CH₂I, NaHCO₃. R_{f}=fluoroalkyl.

FIG. 13 depicts a schematic representations of certain embodiments of static cell adhesion assays.

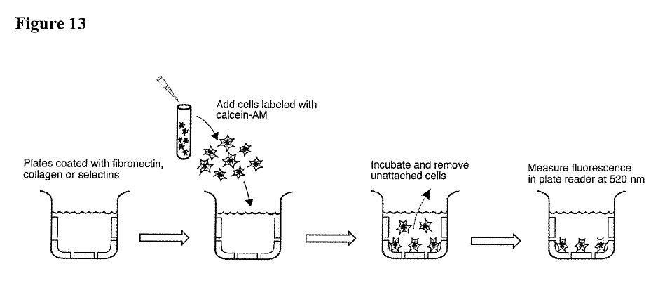

FIG. 14 depicts various N-acyl mannosamines.

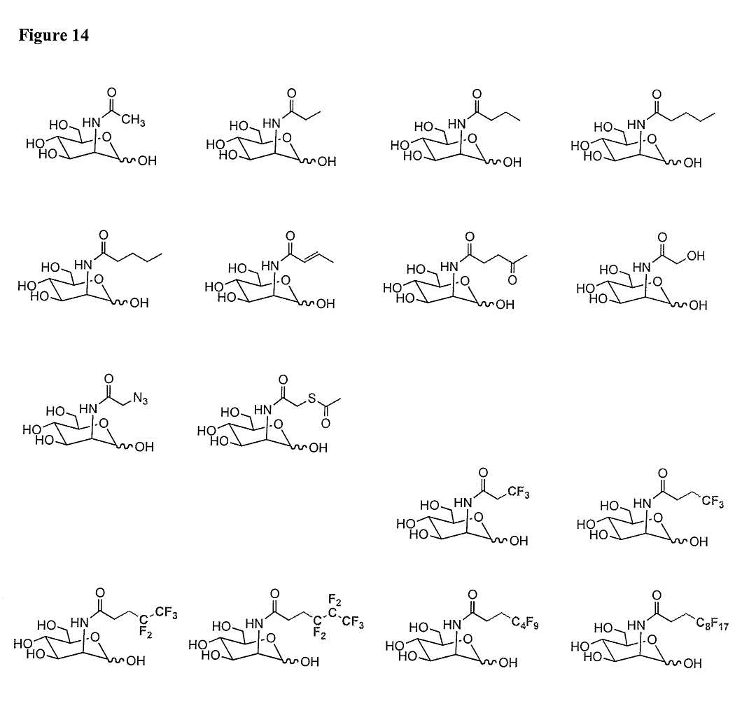

FIG. 15 depicts incorporation of carbohydrates and expression on a cell surface.

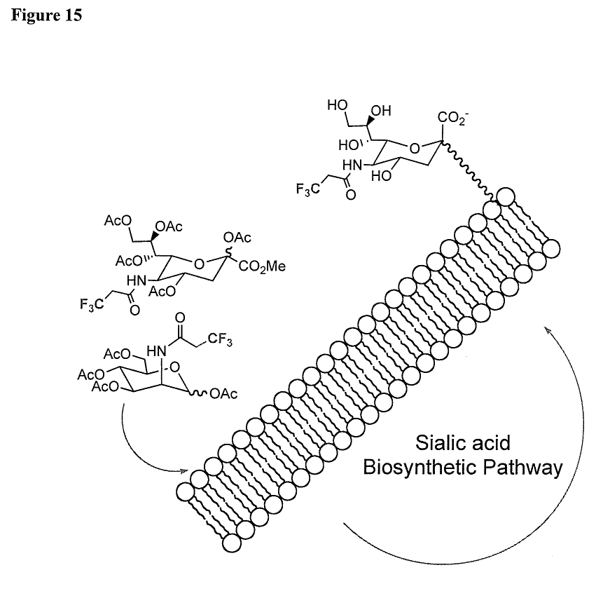

FIG. 16 depicts the fluorescence-based detection of SiaC₄F₃ in HL60 cells.

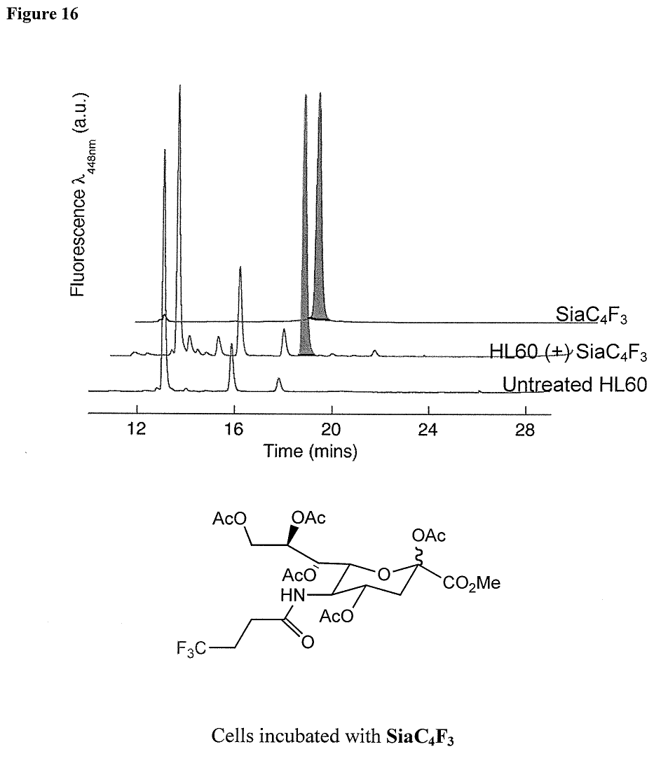

FIG. 17 depicts the fluorescence-based detection of SiaC₅F₃ and ManC₅F₃ in HeLa cells.

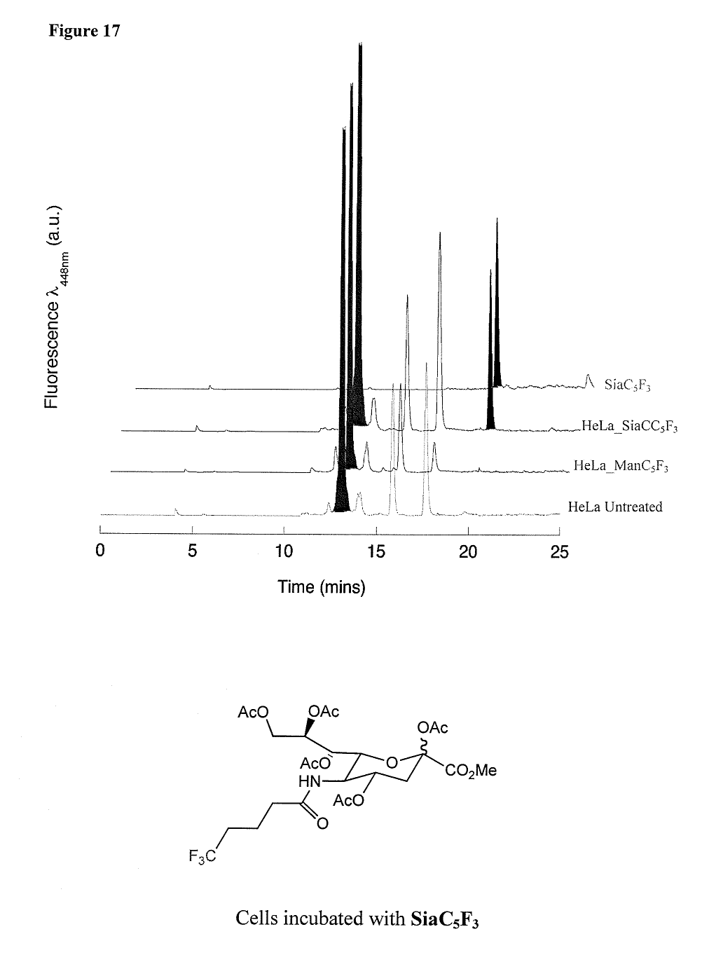

FIG. 18 depicts the fluorescence-based detection of SiaC₅F₃ in HL60 and Jurkat cells.

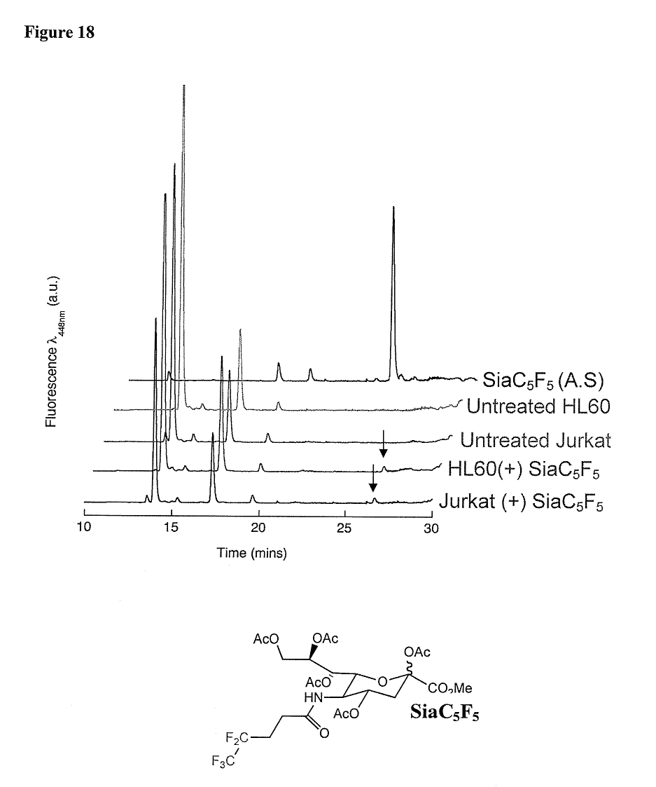

FIG. 19 depicts the fluorescence-based detection of SiaC₆F₇ in HL60 cells and the cytosol.

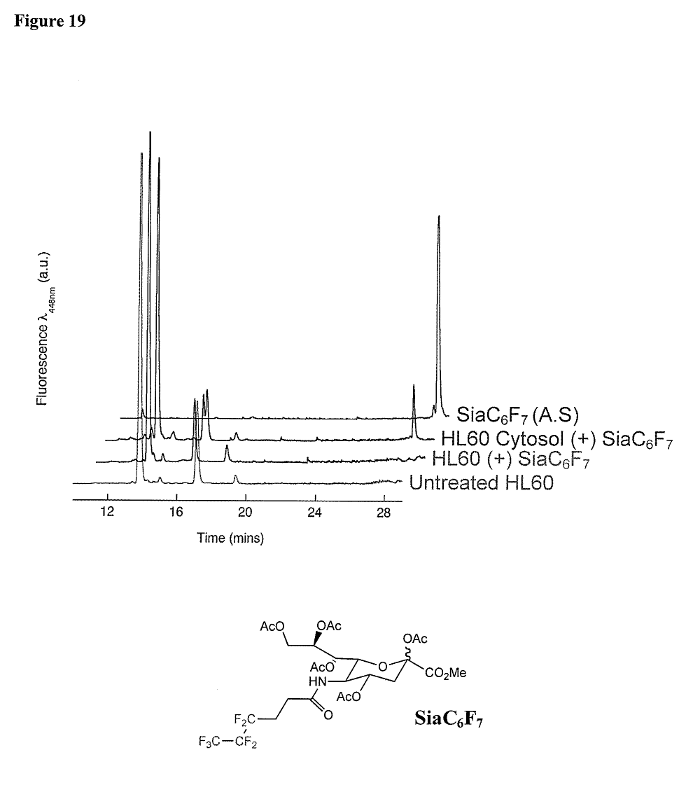

FIG. 20 depicts percent incorporation of mannosamines and sialic acids of the invention.

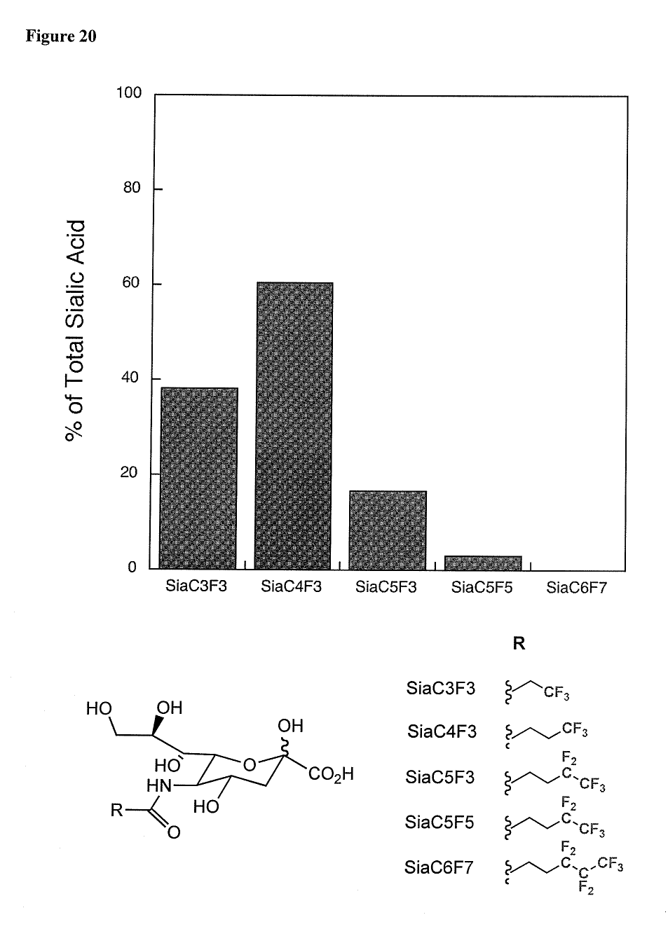

### DETAILED DESCRIPTION OF THE INVENTION

### Definitions

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “binding” can involve any hydrophobic, non-specific, or specific interaction, and the term “biological binding” refers to the interaction between a corresponding pair of molecules that exhibit mutual affinity or binding capacity, typically specific or non-specific binding or interaction. Biological binding defines a type of interaction that occurs between pairs of molecules including proteins, nucleic acids, glycoproteins, carbohydrates, hormones, and the like. Specific examples include protein/carbohydrate, antibody/antigen, antibody/hapten, biotin/streptavidin, biotin/avidin, enzyme/substrate, enzyme/inhibitor, enzyme/cofactor, protein/substrate, lectin/carbohydrate, receptor/hormone, receptor/effector, complementary strands of nucleic acid (e.g., DNA and/or RNA), protein/nucleic acid, repressor/inducer, ligand/receptor, virus/ligand, etc.

As used herein, a “biological entity,” is an entity deriving at least partially from a biological source. Non-limiting examples of biological entities include proteins, peptides, nucleic acids (e.g., oligonucleotides, which may include DNA and/or RNA), fatty acids, carbohydrates, sugars, hormones, enzymes, receptors, lipids, viruses, bacteria, cells, and the like. In some cases, the biological entity has the capability for reproduction, which can be self-reproduction, i.e., a biological entity is a cell (e.g., a bacterium) or a virus. In certain cases, the biological entity is a “pathogen,” i.e., an entity capable of causing a disease when introduced into a subject, for example, a human, a dog, a cat, a horse, a cow, a pig, a sheep, a goat, a chicken, a primate, a rat, a mouse, etc.

As used herein, a “biological recognition element” is an entity able to interact with the biological entity and/or a species present on a biological entity, such as a bacterium, a cell, a virus, etc, for example, by specifically binding to the species. In some cases, the interaction may be a specific interaction. For example, the entity may interact with the species such that the entity has an affinity to the species greater than the affinity of the entity to other species present on the biological entity, or present on similar biological entities. For instance, the biological recognition element may interact with a protein expressed on the surface of a bacterium or a cell, e.g., by binding to the protein, while the biological recognition element does not interact (and/or interacts with less affinity) to other, similar proteins present on the bacterium or cell and/or other bacteria or cells.

In certain cases, the biological recognition element specifically interacts with the biological entity, i.e., the biological recognition element interacts with a particular biological entity (or biological entity type), to a significantly greater degree than to other biological entity. Non-limiting examples of species that may be present on a biological entity include proteins, for example, a cell surface receptor, an enzyme, a structural protein, etc. Other examples include certain receptors and lipids, for instance, phospholipids. An example of a biological recognition element are carbohydrates, for instance, which may specifically bind a protein on the surface of a bacterium or a cell. Examples of carbohydrates include monosaccharides, oligosaccharides, and polysaccharides. Other, non-limiting examples of biological recognition elements include glycosaminoglycans, glycolipids, proteins, antibodies, glycoproteins, and lectins (i.e., glycoproteins able to bind carbohydrates, in some cases, resulting in cell agglomeration). Additional, non-limiting examples of carbohydrates able to bind to biological entities include mannose (which is able to bind Escherichia coli or Salmonella entrica), flicose (which is able to bind Psuedomonas aerginosa), sialic acid (which is able to bind the influenza virus), heparin (which is able to bind herpes simplex virus), or the Lewis group antigens (which are able to bind Helicobacter pylori). In many cases, these interactions are multivalent in nature. In some cases, the carbohydrate may be specifically chosen to bind to a certain biological entity. [Non-limiting examples of such carbohydrates include those discussed in Ratner et al. [Ratner, D. M. “Probing Protein-Carbohydrate Interactions with Microarrays of Synthetic Oligosaccharides” ChemBioChem 2004, 5, 379-383; incorporated by reference in its entirety.]

As used herein, a “carbohydrate” (or, equivalently, a “sugar”) is a saccharide (including monosaccharides, oligosaccharides and polysaccharides) and/or a molecule (including oligomers or polymers) derived from one or more monosaccharides, e.g., by reduction of carbonyl groups, by oxidation of one or more terminal groups to carboxylic acids, by replacement of one or more hydroxy group(s) by a hydrogen atom, an amino group, a thiol group or similar heteroatomic groups, etc. The term “carbohydrate” also includes derivatives of these compounds. Non-limiting examples of carbohydrates include allose (“All”), altrose (“Alt”), arabinose (“Ara”), erythrose, erythrulose, fructose (“Fru”), fucosamine (“FucN”), fucose (“Fuc”), galactosamine (“GalN”), galactose (“Gal”), glucosamine (“GlcN”), glucosaminitol (“GlcN-ol”), glucose (“Glc”), glyceraldehyde, 2,3-dihydroxypropanal, glycerol (“Gro”), propane-1,2,3-triol, glycerone (“1,3-dihydroxyacetone”), 1,3-dihydroxypropanone, gulose (“Gul”), idose (“Ido”), lyxose (“Lyx”), mannosamine (“ManN”), mannose (“Man”), psicose (“Psi”), quinovose (“Qui”), quinovosamine, rhanmitol (“Rha-ol”), rhamnosamine (“RhaN”), rhamnose (“Rha”), ribose (“Rib”), ribulose (“Rul”), sialic acid (“Sia” or “Neu”), sorbose (“Sor”), tagatose (“Tag”), talose (“Tal”), tartaric acid, erythraric/threaric acid, threose, xylose (“Xyl”), or xylulose (“Xul”). In some cases, the carbohydrate may be a pentose (i.e., having 5 carbons) or a hexose (i.e., having 6 carbons); and in certain instances, the carbohydrate may be an oligosaccharide comprising pentose and/or hexose units, e.g., including those described above.

A “monosaccharide,” is a carbohydrate or carbohydrate derivative that includes one saccharide unit. Similarly, a “disaccharide,” a “trisaccharide,” a “tetrasaccharide,” a “pentasaccharide,” etc. respectively has 2, 3, 4, 5, etc. saccharide units. An “oligosaccharide,” as used herein, has 1-20 saccharide units, and the saccharide units may be joined in any suitable configuration, for example, through alpha or beta linkages, using any suitable hydroxy moiety, etc. The oligosaccharide may be linear, or branched in certain instances. A “polysaccharide,” as used herein, typically has at least 4-20 saccharide units. For instance, the polysaccharide may have at least 25 saccharide units, at least 50 saccharide units, at least 75 saccharide units, at least 100 saccharide units, etc. In some cases, the carbohydrate is mulitmeric, i.e., comprising more than one saccharide chain.

For purposes of this invention, the chemical elements are identified in accordance with the Periodic Table of the Elements, CAS version, Handbook of Chemistry and Physics, 67th Ed., 1986-87, inside cover.

The prefix “fluoro” as used herein refers to an alkyl, alkenyl, aryl, aralkyl, or acyl moiety as described below, wherein some or all of the hydrogens have been replaced with fluorines. In certain embodiments, greater than about 80% of the hydrogens have been replaced with fluorines. In certain embodiments, greater than about 90% of the hydrogens have been replaced with fluorines. In certain embodiments, greater than about 95% of the hydrogens have been replaced with fluorines. In certain embodiments, about 100% of the hydrogens have been replaced with fluorines. In certain embodiments, the prefix “fluoro” as applied to an alkyl moiety refers to, for example, an alkyl group represented by “—(CH₂)ₙ(CF₂)ₘCF₃”, wherein n is an integer in range 1 to 10; and m is an integer in range 1 to 10.

The term “perfluoroalkyl” is art-recognized and refers to an alkyl group in which all hydrogens have been replaced with fluorines. For example, trifluoromethyl and pentafluoroethyl are perfluoroalkyl groups.

The term “heteroatom” is art-recognized and refers to an atom of any element other than carbon or hydrogen. Illustrative heteroatoms include boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₃₀ for straight chain, C₃-C₃₀ for branched chain), and more preferably 20 of fewer. Likewise, preferred cycloalkyls have from 4-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

The terms “alkenyl” and “alkynyl” refer to unsaturated aliphatic groups analogous in length and possible substitution to the alkyls described above, but which contain at least one double or triple carbon-carbon bond, respectively.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and includes to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes radicals of polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls, and the radical is on the aromatic ring.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

As used herein, the term “amino” means —NH₂; the term “nitro” means —NO₂; the term “halogen” designates —F, —Cl, —Br or —I; the term “thiol” means —SH; the term “hydroxyl” means —OH; the term “sulfonyl” means —SO₂—; and the term “organometallic” refers to a metallic atom (such as mercury, zinc, lead, magnesium or lithium) or a metalloid (such as silicon, arsenic or selenium) which is bonded directly to a carbon atom, such as a diphenylmethylsilyl group.

The terms “amine” and “amino” are art-recognized and refer to both unsubstituted and substituted amines, e.g., a moiety that may be represented by the general formulas:

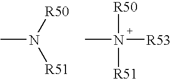

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acyl” refers to C(═O)R62, where R62 is alkyl, cycloalkyl, heterocycloalkyl, alkenyl, alkynyl, aryl, heteroaryl, aralkyl, or heteroaralkyl.

The term “acylamino” is art-recognized and refers to a moiety that may be represented by the general formula:

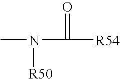

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

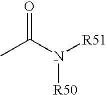

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

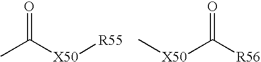

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

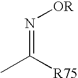

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

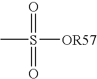

in which R57 is an electron pair, hydrogen, alkyl, cycloalkyl, or aryl.

The term “sulfate” is art recognized and includes a moiety that may be represented by the general formula:

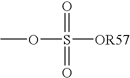

in which R57 is as defined above.

The term “sulfonamido” is art recognized and includes a moiety that may be represented by the general formula:

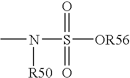

in which R50 and R56 are as defined above.

The term “sulfamoyl” is art-recognized and refers to a moiety that may be represented by the general formula:

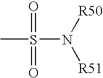

in which R50 and R51 are as defined above.

The term “sulfonyl” is art-recognized and refers to a moiety that may be represented by the general formula:

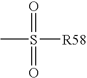

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

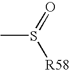

in which R58 is defined above.

The term “phosphoryl” is art-recognized and may in general be represented by the formula:

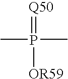

wherein Q50 represents S or O, and R59 represents hydrogen, a lower alkyl or an aryl. When used to substitute, e.g., an alkyl, the phosphoryl group of the phosphorylalkyl may be represented by the general formulas:

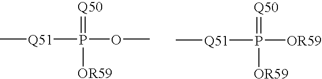

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

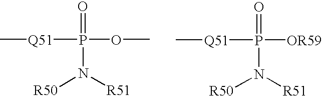

wherein Q51, R50, R51 and R59 are as defined above.

The term “phosphonamidite” is art-recognized and may be represented in the general formulas:

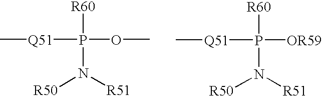

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The definition of each expression, e.g., alkyl, m, n, and the like, when it occurs more than once in any structure, is intended to be independent of its definition elsewhere in the same structure.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

The term “pharmaceutically acceptable salt” or “salt” refers to a salt of one or more compounds. Suitable pharmaceutically acceptable salts of compounds include acid addition salts which may, for example, be formed by mixing a solution of the compound with a solution of a pharmaceutically acceptable acid, such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. Where the compounds carry one or more acidic moieties, pharmaceutically acceptable salts may be formed by treatment of a solution of the compound with a solution of a pharmaceutically acceptable base, such as lithium hydroxide, sodium hydroxide, potassium hydroxide, tetraalkylammonium hydroxide, lithium carbonate, sodium carbonate, potassium carbonate, ammonia, alkylamines, or the like.

The term “pharmaceutically acceptable acid” refers to inorganic or organic acids that exhibit no substantial toxicity. Examples of pharmaceutically acceptable acids include, but are not limited to hydrochloric acid, hydrobromic acid, sulfuric acid, nitric acid, phenylsulfonic acid, methanesulfonic acid, fumaric acid, maleic acid, succinic acid, benzoic acid, acetic acid, citric acid, tartaric acid, phosphoric acid, carbonic acid, and the like.

The term “patient” as used herein, refers to an animal, typically a mammal or a human, that will be or has been the object of treatment, observation, and/or experiment. When the term is used in conjunction with administration of a compound or drug, then the subject has been the object of treatment, observation, and/or administration of the compound or drug.

The term “therapeutically effective amount” as used herein, means that amount of active compound or pharmaceutical agent that elicits a biological or medicinal response in a cell culture, tissue system, animal, or human that is being sought by a researcher, veterinarian, clinician, or physician, which includes alleviation of the symptoms of the disease, condition, or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

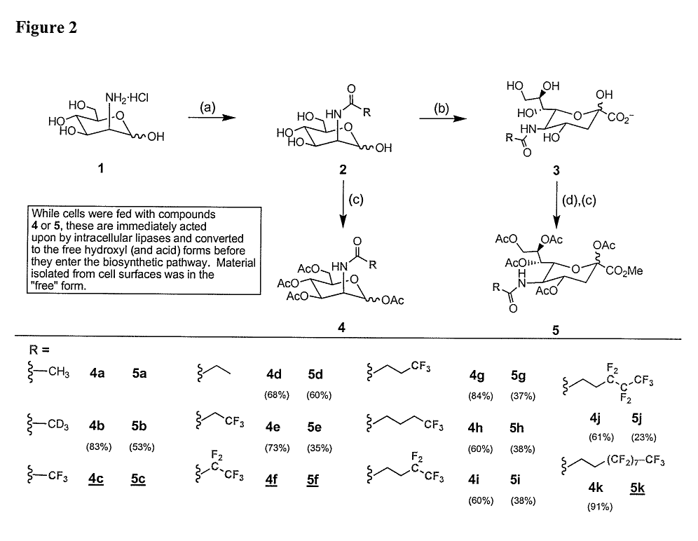

Synthesis of Metabolic Precursors. In one embodiment, fluorinated substrates can be introduced into the sialic acid biosynthetic pathway via controlled fluorination of mannosamine at the C2-position. In certain embodiments, this fluorination is in the form of an N-fluoroacyl side chain. The compounds were prepared in a routine fashion by acylation of the amino group using HBTU as coupling reagent, and further peracetylated using Ac₂O. FIG. 2 shows substrates that have been or can be prepared and tested for their ability to cross the plasma membrane and for subsequent conversion to the corresponding sialic acids by assaying the cytosolic fraction of cell lysates. One can interrogate the ability of the cellular machinery to append these derivatives to cell surface proteins and lipids by hydrolysis and fluorescence labeling of the membrane fraction. All compounds were or may be synthesized in both the free and peracetyl forms. It has been previously demonstrated that the peracetylated derivatives cross the cell membrane easily and are therefore incorporated with higher efficiency. [Jacobs, C. L.; Yarema, K. J.; Mahal, L. K.; Nauman, D. A.; Charters, N. W.; Bertozzi, C. R. In Applications of Chimeric Genes and Hybrid Proteins Pt B 2000; Vol. 327, p 260-75.] The bottleneck step in the biosynthetic pathway for conversion of mannosamine to sialic acids is the phosphorylation of the hydroxyl group at C6 by ManNAc 6-kinase. Because of the relatively strict structural requirements of this enzyme, in certain embodiments the unnatural sialic acids are used directly in feeding experiments. The modified sialic acids can either be prepared chemically, or by enzymatic conversion of N-acyl mannosamines by sialic acid aldolase (E. C. 4.1.3.3) which catalyzes the reversible condensation of N-acetyl-D-mannosamine and pyruvate. [Lin, C. C.; Lin, C. H.; Wong, C. H. “Sialic acid aldolase-catalyzed condensation of pyruvate and N-substituted mannosamine: A useful method for the synthesis of N-substituted sialic acids” Tetrahedron Lett. 1997, 38, 2649-52.] The latter method has been used to obtain fluorinated sialic acids 5e-j (peracetylated and methyl ester of carboxylic acid) in yields ranging from 23-35% over three steps from 2. These compounds were then tested for incorporation on cell surfaces as sialoglycoconjugates.

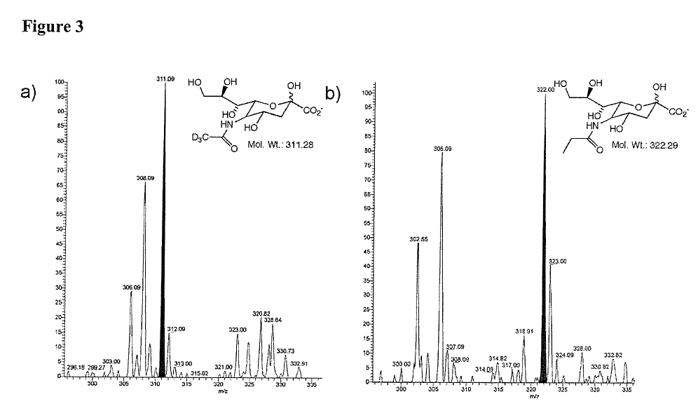

ESI-MS Analysis of Cytosolic Fractions. The ability of fluorinated N-acyl mannosamines 4a, 4b, 4d, 4e, and 4g (see FIG. 2 for structures) to cross the plasma membrane and undergo subsequent conversion to the corresponding sialic acids was probed using electrospray ionization mass spectral (ESI-MS) analysis of cytosolic fractions. Three cell lines, Jurkat (ATTC TIB-152), HL-60 (ATCC CCL-240) and HeLa (ATCC CCL-2) were investigated. Briefly, cells (usually 5×10⁵ cells/mL in 20 mL culture medium) were treated with 200 μM compound for 36-48 hrs, and then the cytosolic fraction was collected by pelleting the cells, washing, and subsequent lysing induced by freeze-thaw cycles. The isolate was then passed through a 3000 MW cutoff filter, concentrated, and analyzed by LC ESI-MS using a Vydac C4 (2.1 mm×150 mm, 5μ) column using a linear gradient of CH₃CN/H₂O. While compounds 5a, 5b and 5d were readily detected and their identities confirmed by co-injection of authentic standards, the fluorinated sialic acids 5e and 5g were not detected in the cytoplasm. FIG. 3 shows the ESI-MS data for cells incubated with compounds 4b and 4d.

Sialic acids do not accumulate in large amounts in the cytoplasm of Jurkat cells. On the other hand the flux going through this structure is large enough to populate the cell surface with 108-1010 sialic acids per cell. Initial findings pointed to two potential reasons for our inability to detect the fluorinated sialic acids in the cytosol: (1) the fluorinated mannosamines are not converted to the corresponding sialic acids; or (2) the sialic acids resulting from biosynthetic conversion are consumed in later steps at a faster rate resulting in a low steady state concentration in the cytosol. These possibilities are discussed below in the context of data obtained from feeding sialic acids directly.

Cell Surface Expression of Fluorinated Sialic Acids. Since the presence of sialic acid analogues in the cytosolic fraction does not reflect cell-surface expression of these molecules, efforts were focused on establishing a reliable assay for sialic acid incorporation on cell-surface sialoglyconjugates. These experiments assay the overall action of multiple biosynthetic steps downstream of sialic acid, namely activation, processing and attachment to proteins and lipids and finally successful trafficking to the membrane. As mentioned above, six derivatives of mannosamine have been prepared and tested. In order to bypass the most stringent step in sialic acid biosynthetic pathway (ManNAc 6-kinase), fluorinated sialic acids were also prepared for direct use in feeding experiments. Sialic acids were synthesized as described above by enzymatic conversion of mannosamines to the corresponding sialic acids. Fluorine containing sialic acids were fed to Jurkat, HeLa and HL-60 cells in the peracetylated, methyl ester form since Reutter and Bertozzi have independently shown that exogenous addition of sialic acids in this form results in efficient incorporation of a wide range of derivatives. The degree of fluorination on the N-acyl chain was varied from a single terminal trifluoromethyl group to a —C₁₁F₁₇ group.

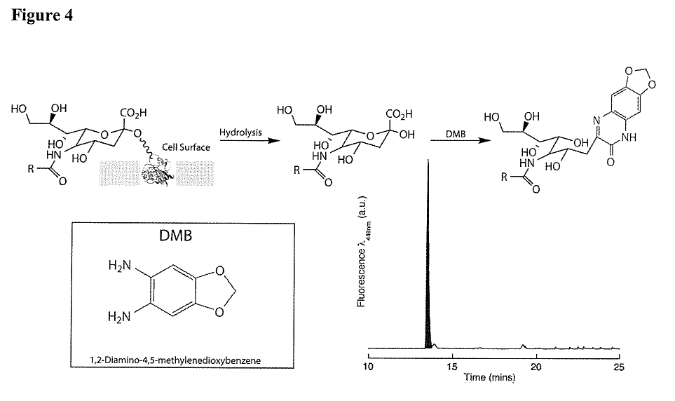

To visualize and quantify the sialic acid incorporation on cell surfaces, 1,2-diamino-4,5-methylenedioxybenzene (DMB) was used as a fluorogenic reagent for labeling α-keto acids (FIG. 4). [Hara, S.; Yamaguchi, M.; Takemori, Y.; Furuhata, K.; Ogura, H.; Nakamura, M. “Determination of Mono-O-Acetylated N-Acetylneuraminic Acids in Human and Rat Sera by Fluorometric High-Performance Liquid-Chromatography” Anal. Biochem. 1989, 179, 162-6.] Briefly, the membrane fraction was obtained after incubation with a given compound (200 μM typical, 900 μM maximum) for 3 days. The membrane pellet was then hydrolyzed by use of 2 M AcOH at 80° C. and then allowed them to react with DMB to yield the corresponding quinoxalinone derivatives. The samples were analyzed by HPLC using a fluorescence detector. The retention times (by comparison to an authentic sample) provide information about the identity, LC ESI-MS unambiguously confirms the molecular structure, and integration of the peaks gives quantitative information on the relative amounts of membrane-derived sialic acids.

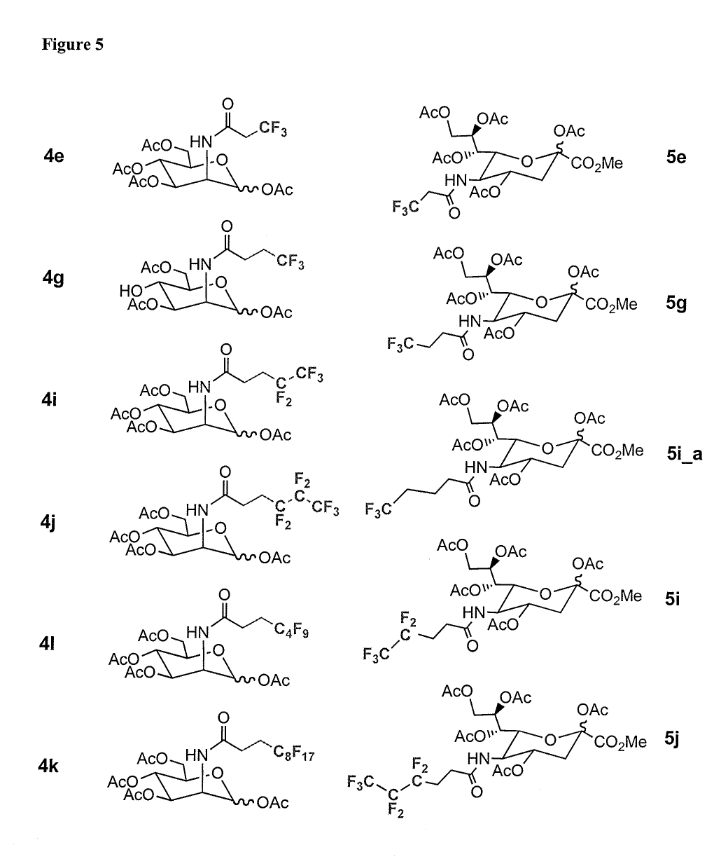

FIG. 5 catalogs mannosamine and sialic acid derivatives tested in this manner. Several groups have explored the limits on acyl chain length and functionality that are permitted by this pathway. The introduction of terminal pentafluoroethyl or trifluoromethyl groups on the acyl chain in the mannosamine derivatives was sometimes sufficient to decrease incorporation significantly compared to the hydrocarbon counterparts. However, sialic acid derivatives were readily incorporated. Certain compounds show reasonably high incorporation efficiencies; for example, 75% of the total sialic acid on the surface of HeLa cells was replaced by 5g.

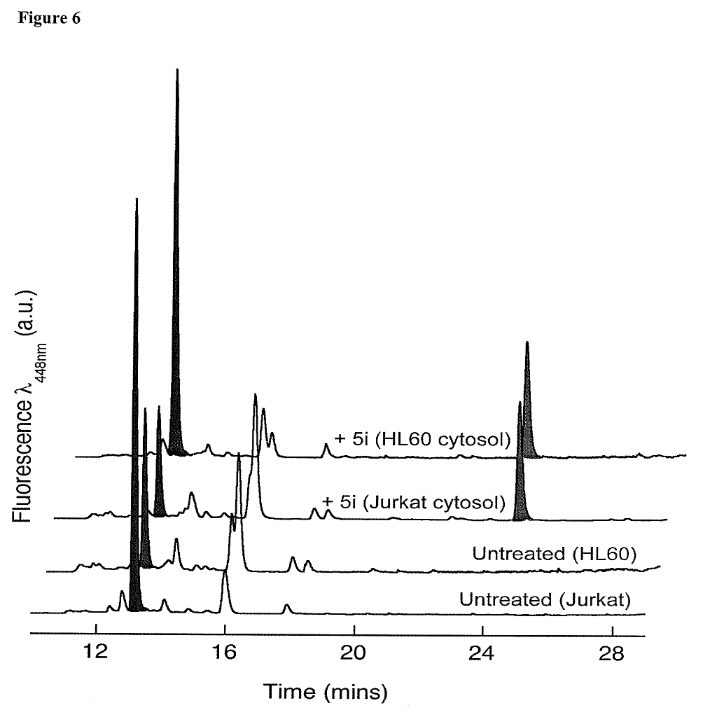

In contrast, when derivative 51 was incubated with either HL60 or Jurkat cells, less than about 5% incorporation was observed. It appears that one of the steps downstream of sialic acid, either CMP activation in the nucleus, or transport to the Golgi, or sialyltransferase action, is compromised. We were able to confirm that membrane passage of 51 in Jurkat or HL60 cells is not diminished since the compound could be isolated from the cytosolic fraction of the cell lysate after 3 days of incubation (FIG. 6).

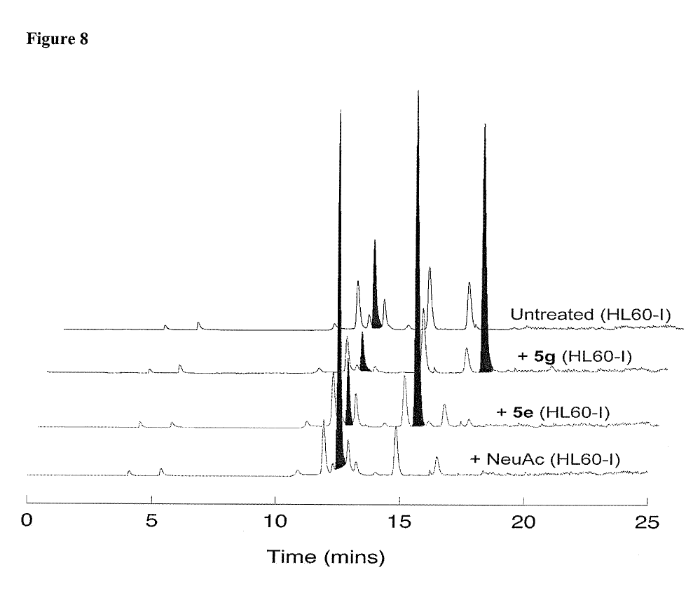

Replacement of Neu5Ac with Unnatural Sialic Acids in Hyposialylated Cells. Cell lines HL60-I and BJA-B K20, variants respectively of the parent HL60 and human B-cell lymphoma (BJA-B), are deficient in UDP-GlcNAc 2-epimerase and show a global suppression of sialylation of glycoconjugates, including expressing mostly unsialylated Lewis-X. [Keppler, 0. T.; Hinderlich, S.; Langner, J.; Schwartz-Albiez, R.; Reutter, W.; Pawlita, M. “UDP-GlcNAc 2-epimerase: A regulator of cell surface sialylation” Science 1999, 284, 1372-6.] Using these hyposialylated cell lines incorporation of fluorinated precursors was investigated and the overall percentage of sialic acids on cell surfaces that are fluorinated was assessed. Both HL60-I and BJA-B K20 cell lines served as metabolic hosts for fluorinated sialic acids 5-N-trifluoropropyl-Neu (5e) and 5-N-trifluorobutyl-Neu (5g) (FIG. 8). Using LC ESI-MS, the incorporation of both derivatives on the surfaces of each cell line was unambiguously confirmed. In contrast, untreated cells only contained Neu5Ac. The endogenous sialic acid in these cells is expressed at levels of about 1×10⁹ molecules per cell, which is about 4-5×10⁹ fluorinated sialic acid molecules on each individual cell.

Furthermore, the degree of incorporation of unnatural sialic acids in cell lines HL60-II which expresses 100-fold larger quantities of sialyl Lewis-X (sLe^{X}) was tested and overall higher degree of sialylation was found; K88 (a sub clone of BJA-B) also exhibited increased sialylation. In either cell line, the fluorinated sialic acids 5-N-trifluoropropyl-Neu (5e) and 5-N-trifluorobutyl-Neu (5g) were again readily incorporated, indicating that the exogenously added sialic acids compete efficiently for sialyltransferase action in the Golgi apparatus. Roughly 75% of the total sialic acid (using substrate 5g) on the cell surface was fluorinated using these cell lines. Taken together these data suggest that a large number of CF₃ groups can be introduced on the surfaces of mammalian cells.

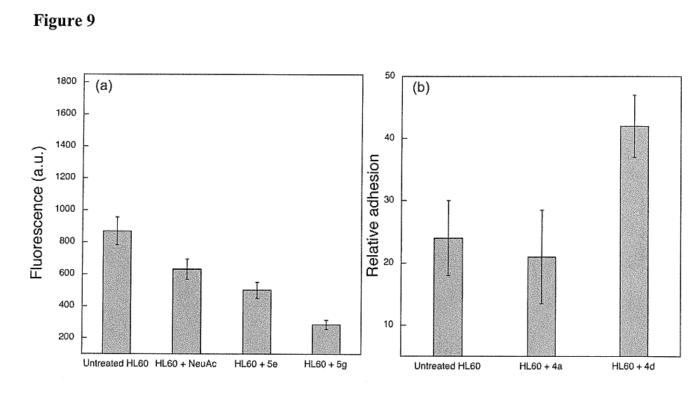

Adhesion of Labeled Cells on Collagen and Fibronectin. The ability of cells labeled with fluorinated carbohydrates to adhere to fibronectin and collagen was also investigated. The adhesive properties of HL60 cells decorated on the surface with either 5-N-trifluoroprop-Neu (5e) or 5-N-trifluorobut-Neu (5g) with those of untreated cells (FIG. 9) were compared. In a typical experiment, cells were incubated with natural or unnatural sialic acids (200 μM) for 72 hours. To quantify adhesion, 96-well plates were coated with fibronectin or collagen (10 μg/mL in PBS) for 16 hours at 4° C. followed by treatment with 1% BSA for 4 hours to block nonspecific adhesion sites. First, cells collected after incubation were suspended in serum free medium for 2 hours. In each of the wells of the 96-well plate, 1×10⁶ cells in 100 μL were introduced and adhesion allowed to proceed at 37° C. for 1 hour. In order to trigger integrin activation and stimulate adhesion of HL60 cells to fibronectin, each well contained 1 nM of phorbol ester PMA or 100 nM of thapsigargin. Unattached cells were washed away by rinsing with PBS and the cells were quantified by addition of the fluorogenic esterase substrate calcein-AM and followed by measurement of emission at 520 nm in a microtiter fluorescence plate reader. Two-tailed Student's t-test were performed on data sets for comparison and only results with P<0.05 were considered significant. When cells were incubated for 72 h in the presence of 5-N-trifluoroprop-Neu (5e), adhesion to fibronectin decreased compared to untreated cells. Furthermore, adhesion of HL60 cells treated with 5-N-trifluorobut-Neu (5g) was decreased to a dramatic extent (˜70%) compared to untreated cells. This change is in the opposite direction to that observed in the case of HL60 cells cultivated in the presence of N-propanoyl-mannosamine (4d); when cells are incubated with 4d, adhesion is increased by more than 130%. [Villavicencio-Lorini, P.; Laabs, S.; Danker, K.; Reutter, W.; Horstkorte, R. “Biochemical engineering of the acyl side chain of sialic acids stimulates integrin-dependent adhesion of HL60 cells to fibronectin” J. Mol. Med. 2002, 80, 671-7; and Horstkorte, R.; Rau, K.; Laabs, S.; Danker, K.; Reutter, W. “Biochemical engineering of the N-acyl side chain of sialic acid leads to increased calcium influx from intracellular compartments and promotes differentiation of HL60 cells” FEBS Lett. 2004, 571, 99-102.] Membrane-bound sialic acid glyconjugates fractions were hydrolyzed and derivatized using 1,2-diamino-4,5-methylenedioxybenzene (DMB) to assess the total amount of sialic acids that had been replaced by the fluorinated counterparts. These results indicate the ability of fluorinated carbohydrates to alter the adhesion properties of eukaryotic cells.

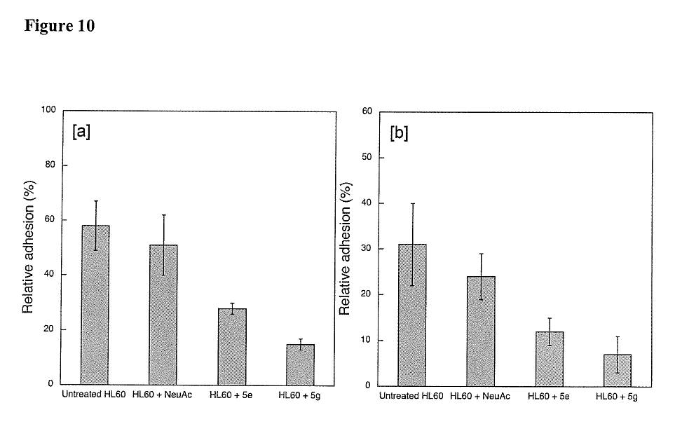

Adhesion of Labeled Cells on E and P selectin-coated plates. The ability of cells labeled with fluorinated carbohydrates to adhere to E and P selectin was investigated using similar static adhesion assays. The adhesive properties of HL60 cells decorated on the surface with either 5-N-trifluoroprop-Neu (5e) or 5-N-trifluorobut-Neu (5g) with those of untreated cells (FIG. 10) were compared. FIG. 10 depicts adhesion of HL60 cells to plates coated with [a] E-selectin (Human CD62E) and [b] P-selectin (Human CD62P). The adhesive properties of HL60 cells decorated on the surface with either 5-N-trifluoroprop-Neu (5e) or 5-N-trifluorobut-Neu (5g) with those of untreated cells (FIG. 9) were compared. In a typical experiment, cells were incubated with natural or unnatural sialic acids for 72 hours, washed with PBS and then allowed to adhere to E and P selectin coated plates. After 1 hr, unattached cells were removed by rinsing with PBS and then the number of cells quantified by addition of calcein-AM and fluorescence counting. These results also indicate the ability of fluorinated carbohydrates to alter the adhesion properties of eukaryotic cells.

The Structural Scope for Analogue Incorporation and Expression on Cultured Cell Surfaces. For exogenously added unnatural sialic acid analogues to be expressed on cell surfaces, they must traverse the cell membrane, undergo esterase hydrolysis of the acetyl groups and the carboxylic ester, enter the nucleus, undergo CMP-Neu5Ac synthetase-catalyzed reaction with CTP, be transported from the nucleus to the ER and/or Golgi, and the resulting modified CMP-sialic acid analogues must be substrates for at least one sialyltransferase. Remarkably, this pathway is quite tolerant of structural variation. In certain embodiments, analogues acylated at the C5 amino group of neuraminic acid with linear fluorinated alkyl acids (e.g., up to 5 carbons long) are capable of passage through the biosynthetic pathway as demonstrated by their appearance on the cell surface. Some of these analogues are already sufficient to alter adhesion characteristics of cells presenting them on their surface.

The ability of sialic acids modified at the 5-position to be expressed on cell surfaces has been studied by several laboratories. The available data suggest that cell-surface presentation decreases dramatically when the alkanoyl group at position 5 reaches 6-7 carbons in length, although an aryl ester is efficiently expressed. Much less is known about the suitability of modifications at other positions and about 5-position modifications that are not amides. Accordingly, in certain embodiments, the incorporation of compounds in FIG. 10 (i.e., compounds with modifications at positions 5 and 9) may be evaluated to determine the extent of their incorporation in cultured HeLa, Jurkat, and HL60 cells.

Compound 51 may be prepared by simple acylation of neuraminic acid followed by methylation of the carboxylic acid and peracetylation. Compound 5m may be synthesized using the fluorous alkylating agent 14 (see FIG. 10). Note that in this case, the sialic acid ethyl ester should be used to avoid the known reaction between 14 and methyl esters.

Compounds 5n-t are fluoroalkylsulfides in which the 9-position hydroxyl group is replaced by various thiofluoroalkyl moieties. These materials may be prepared, as shown in FIG. 10, from known thiol 15 by alkylation of the thiolate under moderately basic conditions with the requisite fluoroalkyl iodides, followed by acetylation. [Martin, R.; Witte, K. L.; Wong, C. H. “The synthesis and enzymatic incorporation of sialic acid derivatives for use as tools to study the structure, activity, and inhibition of glycoproteins and other glycoconjugates” Bioorg. Med. Chem. 1998, 6, 1283-92.] Compound 5u is a 9-position fluoroalkylated amine that will be prepared from known amine 16 using the fluoroalkylating reagent 14. [Fitz, W.; Rosenthal, P. B.; Wong, C. H. “Synthesis and inhibitory properties of a thiomethylmercuric sialic acid with application to the x-ray structure determination of 9-O-acetylsialic acid esterase from influenza C virus” Bioorg. Med. Chem. 1996, 4, 1349-53.] In this case 16 may first be converted to the ethyl ester (step g, FIG. 5) to avoid the side reaction from 14. Final acetylation may provide 5u. Compounds 5v-bb may be prepared by acylation of amine 16, followed by exhaustive acetylation.

Each compound may be incubated with cultured HL60 or HeLa cells and the cell surfaces may be assayed for incorporation of unnatural sialic acids using the DMB-ESI-MS method described below. Compounds may be evaluated in static cell adhesion assays (see below). Compounds in each series (i.e., 5n-t, and 5u-bb) may be synthesized and assessed for incorporation.

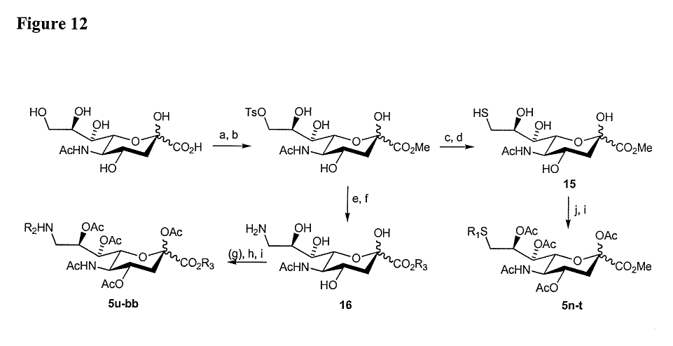

Measurement of Static Adhesion in Fluorinated Sialic Acid Expressing Cells. In certain embodiment, static assays in 96-well plates as described previously may be used to assess the adhesion properties of cells expressing fluorinated sialic acids. For example, cells treated with compounds in FIG. 10 may be first scored for the level of expression of exogenously supplied precursors. In certain embodiments, those cells in which greater than about 10% of the total cell-surface sialic acid is fluorinated may be used in the adhesion assays (FIG. 12).

Cells may be incubated with natural or fluorinated sialic acids (200-900 μM) for 72 hours. To quantify adhesion, 96-well plates coated with fibronectin, collagen, E- or P-selectin can be used. Cells can be allowed to adhere to plates under stimulated (using phorbol ester PMA or thapsigargin in the case of HL60) and unstimulated conditions (HeLa) for 1 hour. Unattached cells can be removed by gentle rinsing with PBS and cells will be quantitated by addition of fluorogenic esterase substrate calcein-AM by measurement of emission at 520 nm in a microtiter fluorescence plate reader. Cells may also be pre-labeled prior to loading on the plate as shown in the FIG. 12. Rigorous Student's t-tests may be performed on data sets to evaluate the statistical significance of the difference in mean values. While these assays provide information about static adhesion to specific molecules, adhesion studies under shear flow conditions can also be performed (see below).

Postulated Mechanism of Altered Adhesion. The mechanism underlying the reduced adhesion of cells expressing fluorinated sialic acids to collagen- or fibronectin-coated surfaces is not yet known. In principle, the change may be due to altered expression or activation of integrins on the cell surface induced by incubation with the fluorinated precursor, or ascribed to differences in binding between the integrin-coated cell when sialic acids are fluorinated, or both. Furthermore, glycoprotein binding interactions may be altered by at least two different mechanisms: altered intermolecular forces due to the presence of the fluorinated group in a region of contact between binding partners or a conformational change in glycoprotein structure induced by the presence of the fluorinated group. Similarly, changes in adhesion of the fluorinated cells to selectin-coated surfaces might be expected if either the sialyl Lewis-X or other sialylated carbohydrates are differently expressed or if the fluorinated sialyl Lewis-X—selectin binding is different from the wild type. It is also possible that the glycoproteins are differently sulfated when fluorinated. To address these issues, both the in vitro binding of model fluorinated carbohydrates to their cognate lectins and the expression of adhesion molecules on the cell surface may be evaluated.

Characterization of Fluorinated Cell Surfaces with Respect to Localization and Distribution: Detecting Modified Sialic Acids on the Cell Surface. Three classes of cell-surface structures are well known to be naturally sialylated: N-linked protein glycans, O-linked protein glycans, and glycolipids. However, while the total amount of cell-surface decoration with a few modified sialic acids has been determined, the distribution of modification among the sialylated molecule classes is unknown. This information is important to develop a molecular level understanding of any phenotypes exhibited by cells displaying fluorinated sialic acids.

Determining the total amount of cell-surface modification by modified sialic acids is straightforward. Briefly, the modified cells are fractionated to separate membrane components; then these are subjected to acid hydrolysis to generate free modified sialic acid that is quantified calorimetrically as its 1,2-diamino-4,5-methylene-dioxybenzene (DMB) derivative. Hara, S.; Takemori, Y.; Yamaguchi, M.; Nakamura, M.; Ohkura, Y. “Fluorometric High-Performance Liquid-Chromatography of N-Acetylneuraminic and N-Glycolylneuraminic Acids and Its Application to Their Microdetermination in Human and Animal Sera, Glycoproteins, and Glycolipids” Anal Biochem. 1987, 164, 138-45. The structure of the detected sialic acid is confirmed by HPLC-ESI-MS analysis compared with synthetic standards (see above).

Effect of Sialic Acid Fluorination on Tumor Metastasis. One embodiment of the invention relates to the correlation of the observed change in the in vitro adhesive properties of cells treated with fluorinated sialic acids with changes in adhesion of cells in an animal leading to significant changes in important biological functions, including immune response and tumor metastasis, suggesting important medicinal applications for the inventive compounds. The case of tumor metastasis is particularly interesting since cells are known to increase their cell-surface expression of sialylated carbohydrates upon cancerous transformation.

In certain embodiments the effect of fluorinated sialic acids on tumor metastasis will be evaluated at two levels: with in vitro rolling, migration, and invasion assays, and in vivo in mouse models for metastasis. In each case an established assay methods may be used to allow direct comparison with other compounds that have been studied previously.

In vitro assays of Cell Rolling, Migration and Invasion. Leukocyte rolling can be measured with an in vitro flow assay. Thoma, G.; Patton, J. T.; Magnani, J. L.; Ernst, B.; Ohrlein, R.; Duthaler, R. O. “Versatile functionalization of polylysine: Synthesis, characterization, and use of neoglycoconjugates” J. Am. Chem. Soc. 1999, 121, 5919-29; and Thoma, G.; Kinzy, W.; Bruns, C.; Patton, J. T.; Magnani, J. L.; Banteli, R. “Synthesis and biological evaluation of a potent E-selectin antagonist” J. Med. Chem. 1999, 42, 4909-13. Native endothelial cells are cultured to form a monolayer on a parallel plate flow cell through which is passed (via syringe pump) a solution containing native polymorphonuclear neutrophils (PMN). The flow cell is positioned on an inverted stage microscope operating in phase-contrast mode and fitted with a CCD camera. A video recorder captures cell-rolling events for later analysis. This technique has been used to evaluate exogenous inhibitors of rolling, but in our case we will first incubate the PMNs with the appropriate precursors to decorate these cells with the fluorinated sialic acids. Expression of the carbohydrates on the cell surface will be quantified with the DMB assay, as described above.

Cell migration may be measured using commercial kits available from Chemicon International, Inc. Briefly, adherent cultured cells are detached from the culture dish with trypsin, then incubated in a Boyden chamber coated with type I collagen or bovine serum albumin. The cells that successfully migrate through the filter are stained and quantified calorimetrically after lysis. Migration is taken as the absorbance due to cells that permeated the collagen-coated filter minus the absorbance due to cells that permeated the BSA-coated negative control. For the instant invention, the cells may be cultured in the presence of fluorinated sialic acids or negative control (either N-acetyl neuraminic acid (Neu5Ac) or vehicle alone prior to measuring migration). Certain experiments may utilize HeLa and differentiated HL60 cells, since efficient cell-surface expression of fluorinated carbohydrates in these cells has been demonstrated, but the assay is general and can be used with other adherent cell lines.

Tumor cell invasion capacity may be assessed using a commercial kit from BD Biosciences. The system is similar to that used in the migration studies, except the coating in the Boyden chamber is the BD Matrigel matrix, a commercial extracellular matrix preparation that models the extracellular basement membrane in that it prevents passage of non-invasive cells. The chamber is loaded with a chemoattractant medium (10% fetal bovine serum) and seeded with the test cells. After incubation, the cells that successfully permeated the matrix are stained and quantified. In certain embodiments, the commonly used invasive tumor cell line, MDA-MB-231 (human breast adenocarcinoma), may be used because it is known to demonstrate invasion in this assay and it is a model that we can use in whole animals for comparison (vide infra). Cultured MDA-MB-231 cells may be incubated with fluorinated sialic acid precursors and expression of the carbohydrates on the cell surface will be quantified with the DMB assay prior to performing the invasion assay.

In Vivo Assays of Tumor Metastasis. Assessing the effect of fluorinated carbohydrates on tumor metastasis is complicated by several factors. Incubation of the metastatic tumor cell line with the fluorinated carbohydrate precursor prior to implantation in an animal is unlikely to provide useful information because the timeframe for metastasis in the mouse models is 4-6 weeks, but the turnover of cell-surface carbohydrates is likely to be on the order of days. Therefore, it is unlikely that an appreciable amount of fluorinated carbohydrates would remain on the tumor cells for the duration of the experiment. Furthermore, tumor growth would dilute the fraction of tumor cells that are labeled.

Therefore, a better strategy is to treat with the fluorinated sialic acids whole animals in which a tumor has been implanted. This approach has the further virtue of mimicking a therapeutic protocol that might prove medically useful. The tissue culture work that has been done so far demonstrates that the compounds are essentially non-toxic at the cell level, but there is no information available regarding the toxicity of the compounds in whole animals.

One system to be studied is the 4T1 metastatic breast cancer model. Aslakson, C. J.; Miller, F. R. “Selective Events in the Metastatic Process Defined by Analysis of the Sequential Dissemination of Subpopulations of a Mouse Mammary-Tumor” Cancer Res. 1992, 52, 1399-405. Nude BALB/c mice will be used initially to decrease the time to metastasis and to reduce any potential immunological complications. The study may be repeated in normal mice. The metastasis model is based on 4T1-luc12B, a luciferase-expressing clone of the 4T1 cell line. Orthotopic introduction of these cells into the mouse mammary fatpad produces a primary tumor that forms metastases in lungs, liver, bone and brain over a period of 3-6 weeks.

Bioluminescent imaging of the mouse using, for example, a Xenogen IVIS 200 Biophotonic Imager, permits monitoring of the growth and metastasis of the tumor. Since chemiluminescence occurs only in the luciferase-expressing tumor cells, there is very low background and thus high sensitivity. The Xenogen instrument permits two-dimensional and pseudo-three-dimensional imaging, the latter based on the spectrum of the emitted light that is altered by absorption of chromophores in the intervening tissue and so is dependent on depth. The optimum dosage of the fluorinated sialic acid in mice will eventually need to be determined empirically. However, Bertozzi's laboratory has demonstrated satisfactory cell-surface expression of an azide-modified sialic acid in live mice by intraperetoneal injection of a modified peracetylated mannosamine precursor once per day for seven days at 300 mg·kg⁻¹ dissolved in 70% aqueous DMSO. That study also demonstrated that plasma esterases did not diminish the incorporation despite the fact that peracetylated precursors were used.

In one experiment, ten mice may be orthotopically injected with 4T1-luc12B cells in the initial study. Half may be treated with compound 5d, the peracetylated trifluoroproponyl neuraminic acid methyl ester with continued treatment for the duration of the experiment. The control animals will be treated with 70% aqueous DMSO on the same regiment. The animals may be imaged and the number and size of metastatic tumors will be scored. Selected treated animals will also be sent for ¹⁹F MRI imaging (vide infra).

The study may be repeated with the MDA-MB-231-luc-D3H1 luciferase-expressing human breast adenocarcinoma line. Jenkins, D. E.; Hornig, Y. S.; Oei, Y.; Dusich, J.; Purchio, T. “Bioluminescent human breast cancer cell lines that permit rapid and sensitive in vivo detection of mammary tumors and multiple metastases in immune deficient mice” Breast Cancer Res. 2005, 7, R444-R54. This study would allow direct comparison of the in vivo metastasis study with the in vitro invasion assay (vide supra).

Use of ¹⁹F MRI to Visualize In Vivo Localization of Cells with Fluorinated Precursors. ¹H and ¹⁹F Magnetic Resonance Imaging (MRI) may be used to visualize the distribution of fluorinated precursors expressed on cell surfaces. Mason, R. P. “Transmembrane pH gradients in vivo: Measurements using fluorinated vitamin B6 derivatives” Curr. Med. Chem. 1999, 6, 481-99; Mason, R. P.; Bansal, N.; Babcock, E. E.; Nunnally, R. L.; Antich, P. P. “A Novel Editing Technique for F-19 Mri-Molecule-Specific Imaging” Magn. Reson. Imaging 1990, 8, 729-36; and Mason, R. P.; Rodbumrung, W.; Antich, P. P. “Hexafluorobenzene: A sensitive F-19 NMR indicator of tumor oxygenation” NMR Biomed. 1996, 9, 125-34. The use of ¹⁹F MRI has been particularly useful because virtually no background is present in biological systems. Venkatasubramanian, P. N.; Shen, Y. J.; Wyrwicz, A. M. “In-Vivo F-19 One-Dimensional Chemical-Shift Imaging Study of Isoflurane Uptake in Rabbit Brain” NMR Biomed. 1993, 6, 377-82; Wolf, W.; Presant, C. A.; Waluch, V. “F-19-MRS studies of fluorinated drugs in humans” Adv. Drug Deliv. Rev. 2000, 41, 55-74; and Yu, J. X.; Kodibagkar, V. D.; Cui, W. N.; Mason, R. P. “F-19: A versatile reporter for non-invasive physiology and pharmacology using magnetic resonance” Curr. Med. Chem. 2005, 12, 819-48. Solid fluorides normally encountered in bones and teeth have very short T₂ relaxation time values resulting in negligible signal. The technique is extremely sensitive to the local environment of the nuclei and MR instruments are easily adapted to the use of a fluorine channel. The model cell line (4T1 metastatic breast cancer model) mentioned above will serve as an initial model for following the fate of fluorinated precursors administered at 300 mg·kg⁻¹.

The MR measurements may be performed on a 4.7 T horizontal bore Varian Unity INOVA system with tunable ¹H/¹⁹F coils and both proton and fluorine images will be acquired sequentially. The presence of trifluoromethyl or higher levels of fluorination will help in increasing signal-to-noise (SNR) ratio. A chemical shift selective imaging mode will be employed in the beginning, but later studies can include relaxation time dependent images. While the rate of clearance of fluorinated carbohydrate precursors from tissues and whole blood are yet to be established and because of the low level of sensitivity of MR techniques, the higher dose value as mentioned previously will serve as a starting point. Furthermore, continuous dosing at this level (300 mg·kg⁻¹) every day will ensure reasonable levels of fluorinated sialic acids on cell surfaces. The last dose may be administered 24 hours before imaging is carried out. Based on the assumption that expression of fluorinated sialic acids on the surface at the levels of Neu5Ac, it is estimated that the concentration of ¹⁹F nucleus in the tumor will be in the 0.6-6 mM range for substitution with one CF₃ group (the ¹⁹F estimate is based on 10⁹ sialic acid groups per individual cell in a 5 mL volume containing 10⁸-10⁹ cells). Images may be acquired by previously established two dimensional pulse sequences with starting values of TR=100 ms, TE=2-3 ms, TH=15 mm, MA=16×16 and FOV=160 mm. Eppendorf tubes (1.5 mL) filled with fluorinated sialic acids (2-5 mM) will be simultaneously imaged with the mice to optimize parametric control of pulse sequences. The image so obtained in 16×16 format will be zero filled to 256×256 picture elements. For each selective MR image a threshold value will be determined to establish the limit above which signals arising from tissues are reliable. This lower limit of this value will be taken as (S_{B}+2σ_{B}) where both parameters (S_{B}=average background signal; σ_{B}=standard deviation) are evaluated in a large area outside the animal.

Bioluminescent imaging (BLI) with 4T1-luc12B, the luciferase-expressing clone of 4T1 may permit direct monitoring of the growth and metastasis of the tumor. On the other hand, MR images will provide direct information on the tissue distribution and metastatic potential of cell populations that have significant fluorine coverage. The extent of ¹⁹F incorporation in metastatic cells, non-metastatic tumor cells, and normal cells may be compared by comparing the BLI with the MRI. The combination may provide a powerful and unique method of assessing the anti-metastatic potential and fluorine coverage by non-invasive imaging.

Breast Cancer Inhibition Model. In certain embodiments, the ability of the inventive compounds to inhibit breast tumor metastasis in vivo may be determined using five to six-week-old, female, nude Balb/C mice. Each experiment may consist of a control group (untreated or vehicle-treated) and treatment groups, with 5 to 10 mice per group. The test compounds may be administered by intraperetoneal injection once per day for seven days at 300 mg kg⁻¹ dissolved in 70% aqueous DMSO. Orthotopically injection of mouse luciferase-expressing (4T1) or human (MDA-MB-231) tumor xenografts represent an excellent tool for the investigation of the antimetastatic activity of novel agents. Tumors in each mouse may be imaged weekly after implant. Animal weights may also be measured at the same time.

Compounds of the Invention. One embodiment of the invention relates to a compound represented by formula I:

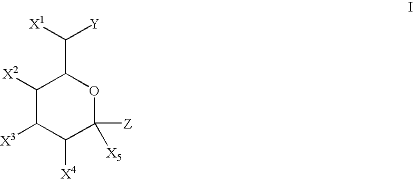

wherein Y is —H or —CHX⁶—CH₂X¹; X¹, X¹, X³, X⁴, X⁵, X⁶, and X⁷ are selected independently from the group consisting of —R¹, —OR², —OC(═O)R³, —NHR⁴, —NHC(═O)R⁵, —SR⁶ and —SC(═O)R⁷; Z is —H or —COOM; M is selected from the group consisting of hydrogen, alkyl, cycloalkyl, heterocycloalkyl, alkenyl, alkynyl, aryl, heteroaryl, aralkyl and heteroaralkyl; R¹, R², R³, R⁴, R⁵, R⁶, and R⁷ are selected independently for each occurrence from the group consisting of hydrogen, alkyl, cycloalkyl, heterocycloalkyl, alkenyl, alkynyl, aryl, heteroaryl, aralkyl, heteroaralkyl, fluoroalkyl, fluorocycloalkyl, fluoroheterocycloalkyl, fluoroalkenyl, fluoroalkynyl, fluoroaryl, fluoroheteroaryl, fluoroaralkyl and fluoroheteroaralky; or any two of R¹, R², R³, R⁴, R⁵, R⁶, and R⁷ on being taken together are —(CH₂)ₙ—; n is independently for each occurrence 1, 2, 3, 4 or 5; and the stereochemical configuration at any stereocenter of a compound represented by I is R, S, or a mixture of these configurations; provided that at least one of R¹, R², R³, R⁴, R⁵, R⁶ or R⁷ is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; or a pharmaceutically acceptable salt thereof.

In certain embodiments, the present invention relates to the aforementioned composition and the attendant definitions, wherein Z is —H.

In certain embodiments, the present invention relates to the aforementioned composition and the attendant definitions, wherein Z is —COOH.

In certain embodiments, the present invention relates to the aforementioned composition and the attendant definitions, wherein Y is H.

In certain embodiments, the present invention relates to the aforementioned composition and the attendant definitions, wherein Y is —CH(OR²)—CH₂X⁷.

In certain embodiments, the present invention relates to the aforementioned composition and the attendant definitions, wherein Y is —CH(OR²)—CH₂OR².

In certain embodiments, the present invention relates to the aforementioned composition and the attendant definitions, wherein X¹, X², X³, X⁴, X⁵, X⁶, and X⁷ are independently —OR², —OC(═O)R³, —NHR⁴, or —NHC(═O)R⁵.

In certain embodiments, the present invention relates to the aforementioned composition and the attendant definitions, wherein X¹, X², X³, X⁴, X⁵, X⁶, and X⁷ are independently —OC(═O)R³ or —NHC(═O)R⁵.

In certain embodiments, the present invention relates to the aforementioned composition and the attendant definitions, wherein X¹, X², X³, X⁴, X⁵, X⁶, and X⁷ are independently —OC(═O)CH₃ or —NHC(═O)R⁵.

In certain embodiments, the present invention relates to the aforementioned composition and the attendant definitions, wherein X¹, X², X³, X⁴, X⁵, X⁶, and X⁷ are independently —OC(═O)CH₃ or —NHC(═O)R⁵; and R⁵ is fluoroalkyl.

In certain embodiments, the present invention relates to the aforementioned composition and the attendant definitions, wherein M is hydrogen.

Another embodiment of the invention relates to a compound represented by formula II:

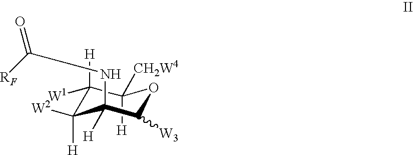

wherein R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; W¹, W², W³, and W⁴ are selected independently from the group consisting of —OR² and —OC(═O)R³; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and n is independently for each occurrence 1, 2, 3, 4 or 5; or a pharmaceutically acceptable salt thereof.

In certain embodiments, the present invention relates to the aforementioned composition and the attendant definitions, wherein R_{F} is fluoroalkyl.

In certain embodiments, the present invention relates to the aforementioned composition and the attendant definitions, wherein R_{F} is perfluoroalkyl.

In certain embodiments, the present invention relates to the aforementioned composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃.

In certain embodiments, the present invention relates to the aforementioned composition and the attendant definitions, wherein W¹, W², W³, and W⁴ are each —OH.

In certain embodiments, the present invention relates to the aforementioned composition and the attendant definitions, wherein W¹, W², W³, and W⁴ are each —OC(═O)CH₃.

In certain embodiments, the present invention relates to the aforementioned composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF—₂)₇CF₃; and W¹, W², W³, and W⁴ are each —OH.

In certain embodiments, the present invention relates to the aforementioned composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF—₂)₇CF₃; and W¹, W², W³, and W⁴ are each —OC(═O)CH₃.

Another embodiment of the invention relates to a compound represented by formula III:

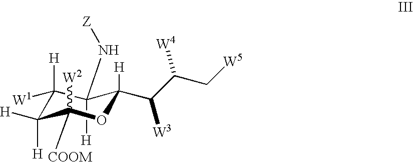

wherein Z is —R_{F} or —C(═O)R_{F}; R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; M is selected from the group consisting of hydrogen, alkyl, cycloalkyl, heterocycloalkyl, alkenyl, alkynyl, aryl, heteroaryl, aralkyl and heteroaralkyl; W¹, W², W³, W⁴ and W⁵ are selected independently from the group consisting of —OR² and —OC(═O)R³; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and n is independently for each occurrence 1, 2, 3, 4 or 5; or a pharmaceutically acceptable salt thereof.

In certain embodiments, the present invention relates to the aforementioned composition and the attendant definitions, wherein Z is —C(═O)R_{F}.

In certain embodiments, the present invention relates to the aforementioned composition and the attendant definitions, wherein Z is —R_{F}.

In certain embodiments, the present invention relates to the aforementioned composition and the attendant definitions, wherein R_{F} is fluoroalkyl.

In certain embodiments, the present invention relates to the aforementioned composition and the attendant definitions, wherein R_{F} is perfluoroalkyl.

In certain embodiments, the present invention relates to the aforementioned composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF—₂)₇CF₃.

In certain embodiments, the present invention relates to the aforementioned composition and the attendant definitions, wherein W¹, W², W³, W⁴ and W⁵ are each —OH.

In certain embodiments, the present invention relates to the aforementioned composition and the attendant definitions, wherein W¹, W², W³, W⁴ and W⁵ are each —OC(═O)CH₃.

In certain embodiments, the present invention relates to the aforementioned composition and the attendant definitions, wherein M is —H.

In certain embodiments, the present invention relates to the aforementioned composition and the attendant definitions, wherein M is —CH₃.

In certain embodiments, the present invention relates to the aforementioned composition and the attendant definitions, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —H.

In certain embodiments, the present invention relates to the aforementioned composition and the attendant definitions, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —CH₃.

Another embodiment of the invention relates to a compound represented by formula IV:

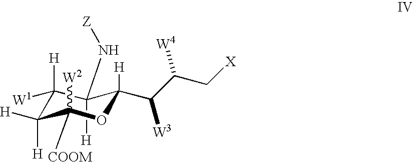

wherein Z is —R_{F} or —C(═O)R³; W¹, W², W³, and W⁴ are selected independently from the group consisting of —OR² and —OC(═O)R³; M is hydrogen, alkyl, cycloalkyl, heterocycloalkyl, alkenyl, alkynyl, aryl, heteroaryl, aralkyl or heteroaralkyl; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; n is independently for each occurrence 1, 2, 3, 4 or 5; X is selected from the group consisting of —R_{F}, —OR_{F}, —OC(═O)R_{F}, —NHR_{F}, —NHC(═O)R_{F}, —SR_{F} and —SC(═O)R_{F}; and R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; or a pharmaceutically acceptable salt thereof.

In certain embodiments, the present invention relates to the aforementioned composition and the attendant definitions, wherein Z is —C(═O)R³.

In certain embodiments, the present invention relates to the aforementioned composition and the attendant definitions, wherein Z is —R_{F}.

In certain embodiments, the present invention relates to the aforementioned composition and the attendant definitions, wherein W¹, W², W³, and W⁴ are each —OH.

In certain embodiments, the present invention relates to the aforementioned composition and the attendant definitions, wherein W¹, W², W³, and W⁴ are each —C(═O)CH₃.

In certain embodiments, the present invention relates to the aforementioned composition and the attendant definitions, wherein M is —H.

In certain embodiments, the present invention relates to the aforementioned composition and the attendant definitions, wherein M is —CH₃.

In certain embodiments, the present invention relates to the aforementioned composition and the attendant definitions, wherein M is —CH₂CH₃.

In certain embodiments, the present invention relates to the aforementioned composition and the attendant definitions, wherein R² and R³ are each alkyl.

In certain embodiments, the present invention relates to the aforementioned composition and the attendant definitions, wherein R² and R³ each —CH₃.

In certain embodiments, the present invention relates to the aforementioned composition and the attendant definitions, wherein R_{F} is fluoroalkyl, fluoroaryl, or fluoroaralkyl.

In certain embodiments, the present invention relates to the aforementioned composition and the attendant definitions, wherein X is —NHR_{F}, —NHC(═O)R_{F}, or —SR_{F}.

In certain embodiments, the present invention relates to the aforementioned composition and the attendant definitions, wherein R_{F} is fluoroalkyl, fluoroaryl, or fluoroaralkyl; and X is —NHR_{F}, —NHC(═O)R_{F}, or —SR_{F}.

In certain embodiments, the present invention relates to the aforementioned composition and the attendant definitions, wherein X is selected from —S(CH₂)₂CF₃, —S(CH—₂)₃CF₃, —S(CH₂)₄CF₃, —SCH₂CF₂CF₃, —SCH₂(CF₂)₂CF₃, —SCH₂(CF₂)₃CF₃, —SCH₂(CF₂)₄CF₃, —NHCH₂C₇F₁₅, —NHC(═O)(CH₂)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, and —NHC(═O)CH₂C₆F₅.

In certain embodiments, the present invention relates to the aforementioned composition and the attendant definitions, wherein Z is —CH₂C₇H₁₅.

In certain embodiments, the present invention relates to the aforementioned composition and the attendant definitions, wherein Z is —CH₂C₇H₁₅; X is —OC(═O)CH₃; W¹, W², W³, and W⁴ are each —C(═O)CH₃; and M is —CH₂CH₃.

In certain embodiments, the present invention relates to the aforementioned composition and the attendant definitions, wherein Z is —C(═O)CH₃; W¹, W², W³, and W⁴ are each —OC(═O)CH₃; M is —CH₃; and X is —S(CH₂)₂CF₃, —S(CH₂)₃CF₃, —S(CH₂)₄CF₃, —SCH₂CF₂CF₃, —SCH₂(CF₂)₂CF₃, —SCH₂(CF₂)₃CF₃, —SCH₂(CF₂)₄CF₃, —NHC(═O)(CH₂)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, or —NHC(═O)CH₂C₆F₅.

In certain embodiments, the present invention relates to the aforementioned composition and the attendant definitions, wherein Z is —C(═O)CH₃; W¹, W², W³, and W⁴ are each —OC(═O)CH₃; M is —CH₂CH₃; and X is —NHCH₂C₇F₁₅.

Another embodiment of the invention relates to polysaccharide and oligosaccharides comprising at least one of the aforementioned composition of formula I, II, III or IV.

Pharmaceutical Compositions. When the compounds of the Formula I, II, III or IV, and their pharmaceutically acceptable salts, are used as antiproliferative agents, such as anticancer agents, or as anti-inflammatory agents, they can be administered to a mammalian subject either alone or in combination with pharmaceutically acceptable carriers or diluents in a pharmaceutical composition according to standard pharmaceutical practice. The compounds can be administered orally or parenterally. Parenteral administration includes intravenous, intramuscular, intraperitoneal, subcutaneous and topical.

Accordingly, the present invention provides pharmaceutically acceptable compositions which comprise a therapeutically-effective amount of one or more of the compounds described above (Formula I-IV), formulated together with one or more pharmaceutically acceptable carriers (additives) and/or diluents. The pharmaceutical compositions of the present invention may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue. The preferred method of administration of compounds of the present invention is parental administration (intravenous).

As set out above, certain embodiments of the present compounds may contain a basic functional group, such as amino or acylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. See, for example, Berge et al “Pharmaceutical Salts”, J. Pharm. Sci. 1977, 66, 1-19.

The pharmaceutically acceptable salts of the compounds of the present invention include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra.)

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite, thioglycerol, sodium mercaptoacetate, and sodium formaldehyde sulfoxylate; (2) oil-soluble antioxidants, such as ascorbyl palmitate, butylated hydroxyanisole (BHA), butylated hydroxytoluene (BHT), lecithin, propyl gallate, alpha-tocopherol.

Examples of pharmaceutically-acceptable buffering agents include, but are not limited to citrate, ascorbate, phosphate, bicarbonate, carbonate, fumarate, acetate, tartarate and malate.

Examples of pharmaceutically-acceptable solubilizing agents include, but are not limited to polyoxyethylene sorbitan fatty acid esters (including polysorbate 80), polyoxyethylene stearates, benzyl alcohol, ethyl alcohol, polyethylene glycols, propylene glycol, glycerin, cyclodextrin, and poloxamers.

Examples of pharmaceutically-acceptable complexing agents include, but are not limited to, cyclodextrins (alpha, beta, gamma), especially substituted beta cyclodextrins such as 2-hydroxypropyl-beta, dimethyl beta, 2-hydroxyethyl beta, 3-hydroxypropyl beta, trimethyl beta.

Examples of pharmaceutically-acceptable metal chelating agents include, but are not limited to, citric acid, ethylenediamine tetraacetic acid (EDTA) and its salt, DTPA (diethylene-triamine-penta-acetic acid) and its salt, EGTA and its salt, NTA (nitriloacetic acid) and its salt, sorbitol and its salt, tartaric acid and its salt, N-hydroxy iminodiacetate and its salt, hydroxyethyl-ethylene diamine-tetraacetic acid and its salt, 1- and 3-propanediamine tetra acetic acid and their salts, 1- and 3-diamino-2-hydroxy propane tetra-acetic acid and their salts, sodium gluconate, hydroxy ethane diphosphonic acid and its salt, and phosphoric acid and its salt.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers (liquid formulation), liquid carriers followed by lyophylization (powder formulation for reconstitution with sterile water or the like), or finely divided solid carriers, or both, and then, if necessary, shaping or packaging the product.

Pharmaceutical compositions of the present invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents. In the examples, the active ingredients are brought together with the pharmaceutically acceptable carriers in solution and then lyophilized to yield a dry powder. The dry powder is packaged in unit dosage form and then reconstituted for parental administration by adding a sterile solution, such as water or normal saline, to the powder.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the compounds of the present invention may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The formulations of the present invention include formulations that are capable of shelf storage as well as formulations used for direct administrations to a patient. Specifically, the pharmaceutical compositions/formulations of the present invention are provide in a form more concentrated than that suitable for direct administration to a patient.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or salt thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable dose of a compound of the invention will be that amount of the compound which is the lowest safe and effective dose to produce a therapeutic effect. Such an effective dose will generally depend upon the factors described above.

While it is possible for a compound of the present invention to be administered alone, it is preferable to administer the compound as a pharmaceutical formulation (composition).

The patient receiving this treatment is any animal in need, including primates, in particular humans, and other mammals such as equines, cattle, swine and sheep; and poultry and pets in general.

One or more other active compounds may be added to the formulations described above to provide formulations for combination therapy.

Compositions and Formulations. The present invention also provides a pharmaceutical composition comprising any one of the aforementioned compounds and at least one pharmaceutically acceptable excipient.

In one embodiment, the present invention provides a pharmaceutical composition comprising: at least one pharmaceutically acceptable excipient; and a compound of formula I:

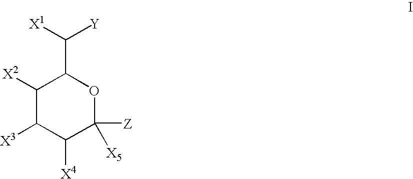

wherein Y is —H or —CHX⁶—CH₂X¹; X¹, X², X¹, X¹, X¹, X⁶ and X⁷ are selected independently from the group consisting of —R¹, —OR², —OC(═O)R³, —NHR⁴, —NHC(═O)R⁵, —SR⁶ and —SC(═O)R⁷; Z is —H or —COOM; M is selected from the group consisting of hydrogen, alkyl, cycloalkyl, heterocycloalkyl, alkenyl, alkynyl, aryl, heteroaryl, aralkyl and heteroaralkyl; R¹, R², R³, R⁴, R⁵, R⁶, and R⁷ are selected independently for each occurrence from the group consisting of hydrogen, alkyl, cycloalkyl, heterocycloalkyl, alkenyl, alkynyl, aryl, heteroaryl, aralkyl, heteroaralkyl, fluoroalkyl, fluorocycloalkyl, fluoroheterocycloalkyl, fluoroalkenyl, fluoroalkynyl, fluoroaryl, fluoroheteroaryl, fluoroaralkyl and fluoroheteroaralky; or any two of R¹, R², R³, R⁴, R⁵, R⁶, and R⁷ on being taken together are —(CH₂)ₙ—; n is independently for each occurrence 1, 2, 3, 4 or 5; and the stereochemical configuration at any stereocenter of a compound represented by I is R, S, or a mixture of these configurations; provided that at least one of R¹, R², R³, R⁴, R⁵, R⁶ or R⁷ is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; or a pharmaceutically acceptable salt thereof.

In certain embodiments, the present invention relates to the aforementioned pharmaceutical composition and the attendant definitions, wherein Z is —H.

In certain embodiments, the present invention relates to the aforementioned pharmaceutical composition and the attendant definitions, wherein Z is —COOH.

In certain embodiments, the present invention relates to the aforementioned pharmaceutical composition and the attendant definitions, wherein Y is H.

In certain embodiments, the present invention relates to the aforementioned pharmaceutical composition and the attendant definitions, wherein Y is —CH(OR²)—CH₂X⁷.

In certain embodiments, the present invention relates to the aforementioned pharmaceutical composition and the attendant definitions, wherein Y is —CH(OR²)—CH₂OR².

In certain embodiments, the present invention relates to the aforementioned pharmaceutical composition and the attendant definitions, wherein X¹, X², X³, X⁴, X⁵, X⁶, and X⁷ are independently —OR², —OC(═O)R³, —NHR⁴, or —NHC(═O)R⁵.

In certain embodiments, the present invention relates to the aforementioned pharmaceutical composition and the attendant definitions, wherein X¹, X², X³, X⁴, X⁵, X⁶, and X⁷ are independently —OC(═O)R³ or —NHC(═O)R⁵.

In certain embodiments, the present invention relates to the aforementioned pharmaceutical composition and the attendant definitions, wherein X¹, X², X³, X⁴, X⁵, X⁶, and X⁷ are independently —OC(═O)CH₃ or —NHC(═O)R⁵.

In certain embodiments, the present invention relates to the aforementioned pharmaceutical composition and the attendant definitions, wherein X¹, X², X³, X⁴, X⁵, X⁶, and X⁷ are independently —OC(═O)CH₃ or —NHC(═O)R⁵ and R⁵ is fluoroalkyl.

In certain embodiments, the present invention relates to the aforementioned pharmaceutical composition and the attendant definitions, wherein M is hydrogen.

In another embodiment, the present invention provides a pharmaceutical composition comprising: at least one pharmaceutically acceptable excipient; and a compound of formula II:

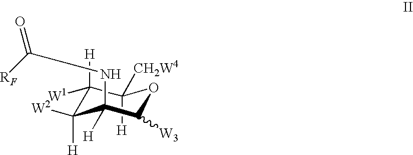

wherein R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; W¹, W², W³, and W⁴ are selected independently from the group consisting of —OR and —OC(═O)R³; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and n is independently for each occurrence 1, 2, 3, 4 or 5; or a pharmaceutically acceptable salt thereof.

In certain embodiments, the present invention relates to the aforementioned pharmaceutical composition and the attendant definitions, wherein R_{F} is fluoroalkyl.

In certain embodiments, the present invention relates to the aforementioned pharmaceutical composition and the attendant definitions, wherein R_{F} is perfluoroalkyl.

In certain embodiments, the present invention relates to the aforementioned pharmaceutical composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃.

In certain embodiments, the present invention relates to the aforementioned pharmaceutical composition and the attendant definitions, wherein W¹, W², W³, and W⁴ are each —OH.

In certain embodiments, the present invention relates to the aforementioned pharmaceutical composition and the attendant definitions, wherein W¹, W², W³, and W⁴ are each —OC(═O)CH₃.

In certain embodiments, the present invention relates to the aforementioned pharmaceutical composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; and W¹, W², W³, and W⁴ are each —OH.

In certain embodiments, the present invention relates to the aforementioned pharmaceutical composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; and W¹, W², W³, and W⁴ are each —OC(═O)CH₃.

In another embodiment, the present invention provides a pharmaceutical composition comprising: at least one pharmaceutically acceptable excipient; and a compound of formula III:

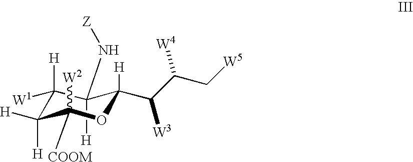

wherein Z is —R_{F} or —C(═O)R_{F}; R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; M is selected from the group consisting of hydrogen, alkyl, cycloalkyl, heterocycloalkyl, alkenyl, alkynyl, aryl, heteroaryl, aralkyl and heteroaralkyl; wherein Z is —R_{F} or —C(═O)R_{F}; R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; M is selected from the group consisting of hydrogen, alkyl, cycloalkyl, heterocycloalkyl, alkenyl, alkynyl, aryl, heteroaryl, aralkyl and heteroaralkyl; W¹, W², W¹, W⁴ and W⁵ are selected independently from the group consisting of —OR² and —OC(═O)R³; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and n is independently for each occurrence 1, 2, 3, 4 or 5; or a pharmaceutically acceptable salt thereof.

In certain embodiments, the present invention relates to the aforementioned pharmaceutical composition and the attendant definitions, wherein Z is —C(═O)R_{F}.

In certain embodiments, the present invention relates to the aforementioned pharmaceutical composition and the attendant definitions, wherein Z is —R_{F}.

In certain embodiments, the present invention relates to the aforementioned pharmaceutical composition and the attendant definitions, wherein R_{F} is fluoroalkyl.

In certain embodiments, the present invention relates to the aforementioned pharmaceutical composition and the attendant definitions, wherein R_{F} is perfluoroalkyl.

In certain embodiments, the present invention relates to the aforementioned pharmaceutical composition and the attendant definitions, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃.

In certain embodiments, the present invention relates to the aforementioned pharmaceutical composition and the attendant definitions, wherein W¹, W², W³, W⁴ and W⁵ are each —OH.

In certain embodiments, the present invention relates to the aforementioned pharmaceutical composition and the attendant definitions, wherein W¹, W², W³, W⁴ and W⁵ are each —OC(═O)CH₃.

In certain embodiments, the present invention relates to the aforementioned pharmaceutical composition and the attendant definitions, wherein M is —H.

In certain embodiments, the present invention relates to the aforementioned pharmaceutical composition and the attendant definitions, wherein M is —CH₃.

In certain embodiments, the present invention relates to the aforementioned pharmaceutical composition and the attendant definitions, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —H.

In certain embodiments, the present invention relates to the aforementioned pharmaceutical composition and the attendant definitions, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —CH₃.

In another embodiment, the present invention provides a pharmaceutical composition comprising: at least one pharmaceutically acceptable excipient; and a compound of formula IV:

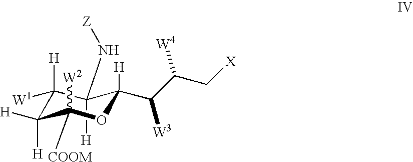

wherein Z is —R_{F} or —C(═O)R³; W¹, W², W³, and W⁴ are selected independently from the group consisting of —OR² and —OC(═O)R³; M is hydrogen, alkyl, cycloalkyl, heterocycloalkyl, alkenyl, alkynyl, aryl, heteroaryl, aralkyl or heteroaralkyl; R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; n is independently for each occurrence 1, 2, 3, 4 or 5; X is selected from the group consisting of —R_{F}, —OR_{F}, —OC(═O)R_{F}, —NHR_{F}, —NHC(═O)R_{F}, —SR_{F} and —SC(═O)R_{F}; and R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl; or a pharmaceutically acceptable salt thereof.

In certain embodiments, the present invention relates to the aforementioned composition and the attendant definitions, wherein Z is —R_{F}.

In certain embodiments, the present invention relates to the aforementioned pharmaceutical composition and the attendant definitions, wherein W¹, W², W³, and W⁴ are each —OH.

In certain embodiments, the present invention relates to the aforementioned pharmaceutical composition and the attendant definitions, wherein W¹, W², W³, and W⁴ are each —C(═O)CH₃.

In certain embodiments, the present invention relates to the aforementioned pharmaceutical composition and the attendant definitions, wherein M is —H.

In certain embodiments, the present invention relates to the aforementioned pharmaceutical composition and the attendant definitions, wherein M is —CH₃.

In certain embodiments, the present invention relates to the aforementioned pharmaceutical composition and the attendant definitions, wherein M is —CH₂CH₃.

In certain embodiments, the present invention relates to the aforementioned pharmaceutical composition and the attendant definitions, wherein R² and R³ are each alkyl.

In certain embodiments, the present invention relates to the aforementioned pharmaceutical composition and the attendant definitions, wherein R² and R³ each —CH₃.

In certain embodiments, the present invention relates to the aforementioned pharmaceutical composition and the attendant definitions, wherein R_{F} is fluoroalkyl, fluoroaryl, or fluoroaralkyl.

In certain embodiments, the present invention relates to the aforementioned pharmaceutical composition and the attendant definitions, wherein X is —NHR_{F}, —NHC(═O)R_{F}, or —SR_{F}.

In certain embodiments, the present invention relates to the aforementioned pharmaceutical composition and the attendant definitions, wherein R_{F} is fluoroalkyl, fluoroaryl, or fluoroaralkyl; and X is —NHR_{F}, —NHC(═O)R_{F}, or —SR_{F}.

In certain embodiments, the present invention relates to the aforementioned pharmaceutical composition and the attendant definitions, wherein X is selected from —S(CH₂)₂CF₃, —S(CH₂)₃CF₃, —S(CH₂)₄CF₃, —SCH₂CF₂CF₃, —SCH₂(CF₂)₂CF₃, —SCH₂(CF₂)₃CF₃, —SCH₂(CF₂)₄CF₃, —NHCH₂C₇F₁₅, —NHC(═O)(CH₂)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, and —NHC(═O)CH₂C₆F₅.

In certain embodiments, the present invention relates to the aforementioned pharmaceutical composition and the attendant definitions, wherein Z is —CH₂C₇H₁₅.

In certain embodiments, the present invention relates to the aforementioned pharmaceutical composition and the attendant definitions, wherein Z is —CH₂C₇H₁₅; X is —OC(═O)CH₃; W¹, W², W³, and W⁴ are each —C(═O)CH₃; and M is —CH₂CH₃.

In certain embodiments, the present invention relates to the aforementioned pharmaceutical composition and the attendant definitions, wherein Z is —C(═O)CH₃; W¹, W², W³, and W⁴ are each —OC(═O)CH₃; M is —CH₃; and X is —S(CH₂)₂CF₃, —S(CH₂)₃CF₃, —S(CH₂)₄CF₃, —SCH₂CF₂CF₃, —SCH₂(CF₂)₂CF₃, —SCH₂(CF₂)₃CF₃, —SCH₂(CF₂)₄CF₃, —NHC(═O)(CH₂)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, or —NHC(═O)CH₂C₆F₅.

In certain embodiments, the present invention relates to the aforementioned pharmaceutical composition and the attendant definitions, wherein Z is —C(═O)CH₃; W¹, W², W³, and W⁴ are each —OC(═O)CH₃; M is —CH₂CH₃; and X is —NHCH₂C₇F₁₅.

### Methods of the Invention.

In one embodiment, the present invention provides a method of reducing cellular adhesion, comprising the steps of:

contacting said cell with a fluorine-containing monosaccharide; and

incubating said cell under conditions whereby the cell internalizes said fluorine-containing monosaccharide and extracellularly expresses a glucoconjugate comprising said fluorine-containing monosaccharide, or a derivative thereof, on the surface of said cell.

In certain embodiments, the present invention relates to the aforementioned method, wherein said fluorine-containing monosaccharide is any one of the aforementioned compounds; or a therapeutically effective amount of any one of the aforementioned pharmaceutical compositions.

In certain embodiments, the present invention relates to the aforementioned method, wherein said cell is selected from the group consisting of B cells, T cells, granulocytes, melanoma cells, breast cancer cells, lymphoma cells, osteosarcoma cells, leukemia cells, squamous carcinoma cells, cervical cancer cells, ovarian cancer cells, pancreatic cancer cells, and fibrosarcoma cells.

In one embodiment, the present invention provides a method of imaging all or part of an organ of a mammal, comprising the steps of

administering to said mammal a detectable amount of a fluorine-containing monosaccharide or composition thereof; and

subjecting said mammal to nuclear magnetic resonance imaging.

In one embodiment, the present invention relates to the aforementioned method, wherein said organ is a liver, a kidney, a heart, skin, a brain, an eye, a pancreas, a stomach, an intestine, a thyroid, a lung, a rectum, a uterus, a cervix, a prostate, a breast, a testicle, a brainstem, or a bladder.

In one embodiment, the present invention relates to the aforementioned method, wherein said mammal is a primate, equine, canine, feline, or bovine.

In one embodiment, the present invention relates to the aforementioned method, wherein said mammal is a human.

In one embodiment, the present invention relates to the aforementioned method, wherein the mode of administration of said fluorine-containing monosaccharide or composition thereof is inhalation, oral, intravenous, sublingual, ocular, transdermal, rectal, vaginal, topical, intramuscular, intra-arterial, intrathecal, subcutaneous, buccal, or nasal.

In one embodiment, the present invention relates to the aforementioned method, wherein the mode of administration is intravenous.

In certain embodiments, the present invention relates to the aforementioned method, wherein said fluorine-containing monosaccharide is any one of the aforementioned compounds; or a therapeutically effective amount of any one of the aforementioned pharmaceutical compositions.

In one embodiment, the present invention provides a method of treating inflammation, comprising administering to a mammal in need thereof a therapeutically effective amount of any one of the aforementioned compounds; or a therapeutically effective amount of any one of the aforementioned pharmaceutical compositions.

In one embodiment, the present invention relates to the aforementioned method, wherein said inflammation is inflammation of the pulmonary system, gastrointestinal system, musculoskeletal system, reproductive system, central nervous system, or urologic system.

In one embodiment, the present invention relates to the aforementioned method, wherein the inflammation is located in the mammal's myeloid tissues, lymphoid tissues, pancreatic tissues, thyroid tissues, lungs, colon tissues, rectal tissues, anal tissues, liver tissues, skin, bone, ovarian tissues, uterine tissues, cervical tissues, breast, prostate, testicular tissues, brain, brainstem, meningial tissues, kidney, or bladder.

In one embodiment, the present invention relates to the aforementioned method, wherein the inflammation is located in the mammal's myeloid tissues, lymphoid tissues, breast, lung, ovary, or prostate.

In one embodiment, the present invention relates to the aforementioned method, wherein said inflammation is caused by cardiovascular disease, chronic obstructive pulmonary disease (COPD), arthritis, rheumatoid arthritis, multiple sclerosis, asthma, inflammatory bowel disease, Crohn's disease, Behcet's disease, allergic rhinitis (hay fever), pelvic inflammatory disease, inflammatory disease of the thyroid, diabetes mellitus, lupus erythematosus, Kawasaki disease, immune thrombocytopenic purpura, necrotizing enterocolitis, nephritis, atherosclerosis, psoriasis, gout, and sarcoidosis.

In one embodiment, the present invention relates to the aforementioned method, wherein said mammal is a human.

In one embodiment, the present invention relates to the aforementioned method, wherein the mode of administration of said compound is inhalation, oral, intravenous, sublingual, ocular, transdermal, rectal, vaginal, topical, intramuscular, intra-arterial, intrathecal, subcutaneous, buccal, or nasal.

In one embodiment, the present invention relates to the aforementioned method, wherein the mode of administration is intravenous.

In one embodiment, the present invention provides a method of treating cancer, comprising administering to a mammal in need thereof a therapeutically effective amount of any one of the aforementioned compounds; or a therapeutically effective amount of any one of the aforementioned pharmaceutical compositions.

In one embodiment, the present invention relates to the aforementioned method, wherein said cancer is a cancer of the hematopoietic system, immune system, endocrine system, pulmonary system, gastrointestinal system, musculoskeletal system, reproductive system, central nervous system, or urologic system.

In one embodiment, the present invention relates to the aforementioned method, wherein the cancer is located in the mammal's myeloid tissues, lymphoid tissues, pancreatic tissues, thyroid tissues, lungs, colon tissues, rectal tissues, anal tissues, liver tissues, skin, bone, ovarian tissues, uterine tissues, cervical tissues, breast, prostate, testicular tissues, brain, brainstem, meningial tissues, kidney, or bladder.

In one embodiment, the present invention relates to the aforementioned method, wherein the cancer is located in the mammal's myeloid tissues, lymphoid tissues, breast, lung, ovary, or prostate.

In one embodiment, the present invention relates to the aforementioned method, wherein said cancer is breast cancer, multiple myeloma, prostate cancer, Hodgkin lymphoma, non-Hodgkin lymphoma, acute lymphocytic leukemia, chronic lymphocytic leukemia, acute myeloid leukemia, chronic myeloid leukemia, renal cell carcinoma, malignant melanoma, pancreatic cancer, lung cancer, colorectal carcinoma, colon cancer, brain cancer, renal cancer, head and neck cancer, bladder cancer, thyroid cancer, prostate cancer, ovarian cancer, cervical cancer, or myelodysplastic syndrome.

In one embodiment, the present invention relates to the aforementioned method, wherein said mammal's cancer is breast cancer, acute myeloid leukemia, chronic myeloid leukemia, melanoma, multiple myeloma, lung cancer, ovarian cancer, or prostate cancer.

In one embodiment, the present invention relates to the aforementioned method, wherein said mammal is a primate, equine, canine, feline, or bovine.

In one embodiment, the present invention relates to the aforementioned method, wherein said mammal is a human.

In one embodiment, the present invention relates to the aforementioned method, wherein the mode of administration of said compound is inhalation, oral, intravenous, sublingual, ocular, transdermal, rectal, vaginal, topical, intramuscular, intra-arterial, intrathecal, subcutaneous, buccal, or nasal.

In one embodiment, the present invention relates to the aforementioned method, wherein the mode of administration is intravenous.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound represented by formula I: 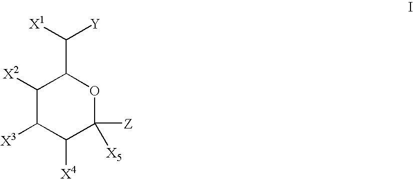
wherein
Y is —H or —CHX⁶—CH₂X⁷;
X¹, X², X³, X⁴, X⁵, X⁶, and X⁷ are selected independently from the group consisting of —R¹, —OR², —OC(═O)R³, —NHR⁴, —NHC(═O)R⁵, —SR⁶ and —SC(═O)R⁷;
Z is —H or —COOM;
M is selected from the group consisting of hydrogen, alkyl, cycloalkyl, heterocycloalkyl, alkenyl, alkynyl, aryl, heteroaryl, aralkyl and heteroaralkyl;
R¹, R², R³, R⁴, R⁵, R⁶, and R⁷ are selected independently for each occurrence from the group consisting of hydrogen, alkyl, cycloalkyl, heterocycloalkyl, alkenyl, alkynyl, aryl, heteroaryl, aralkyl, heteroaralkyl, fluoroalkyl, fluorocycloalkyl, fluoroheterocycloalkyl, fluoroalkenyl, fluoroalkynyl, fluoroaryl, fluoroheteroaryl, fluoroaralkyl and fluoroheteroaralky; or any two of R¹, R², R³, R⁴, R⁵, R⁶, and R⁷ on being taken together are —(CH₂)ₙ—;
n is independently for each occurrence 1, 2, 3, 4 or 5; and
the stereochemical configuration at any stereocenter of a compound represented by I is R, S, or a mixture of these configurations;
provided that at least one of R¹, R², R³, R⁴, R⁵, R⁶ or R⁷ is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl;
or a pharmaceutically acceptable salt thereof.

2. The compound of claim 1, wherein Z is —H.

3. The compound of claim 1, wherein Z is —COOH.

4. The compound of claim 1, wherein Y is H.

5. The compound of claim 1, wherein Y is —CH(OR²)—CH₂X₇.

6. The compound of claim 1, wherein Y is —CH(OR²)—CH₂OR².

7. The compound of claim 1, wherein X¹, X², X³, X⁴, X⁵, X⁶, and X⁷ are independently —OR², —OC(═O)R³, —NHR⁴, or —NHC(═O)R⁵.

8. The compound of claim 1, wherein X¹, X², X¹, X⁴, X⁵, X⁶, and X⁷ are independently —OC(═O)R³ or —NHC(═O)R⁵.

9. The compound of claim 1, wherein X¹, X², X³, X⁴, X⁵, X⁶, and X⁷ are independently —OC(═O)CH₃ or —NHC(═O)R⁵.

10. The compound of claim 1, wherein X¹, X², X³, X⁴, X⁵, X⁶, and X⁷ are independently —OC(═O)CH₃ or —NHC(═O)R⁵; and R⁵ is fluoroalkyl.

11. The compound of claim 1, wherein M is hydrogen.

12. A compound represented by formula II: 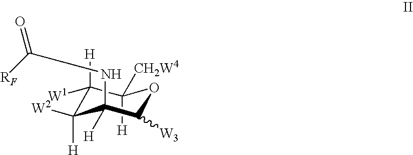
wherein
R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl;
W¹, W², W³, and W⁴ are selected independently from the group consisting of —OR² and —OC(═O)R;
R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and
n is independently for each occurrence 1, 2, 3, 4 or 5;
or a pharmaceutically acceptable salt thereof.

13. The compound of claim 12, wherein R_{F} is fluoroalkyl.

14. The compound of claim 12, wherein R_{F} is perfluoroalkyl.

15. The compound of claim 12, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃.

16. The compound of claim 12, wherein W¹, W², W³, and W⁴ are each —OH.

17. The compound of claim 12, wherein W¹, W², W³, and W⁴ are each —OC(═O)CH₃.

18. The compound of claim 12, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; and W¹, W², W³, and W⁴ are each —OH.

19. The compound of claim 12, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; and W¹, W², W³, and W⁴ are each —OC(═O)CH₃.

20. A compound represented by formula III: 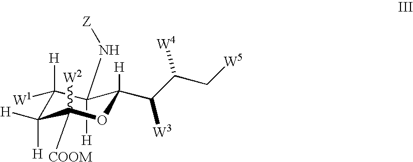
wherein
Z is —R_{F} or —C(═O)R_{F};
R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl;
M is selected from the group consisting of hydrogen, alkyl, cycloalkyl, heterocycloalkyl, alkenyl, alkynyl, aryl, heteroaryl, aralkyl and heteroaralkyl;
W¹, W², W³, W⁴ and W⁵ are selected independently from the group consisting of —OR² and —OC(═O)R³;
R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—; and
n is independently for each occurrence 1, 2, 3, 4 or 5;
or a pharmaceutically acceptable salt thereof.

21. The compound of claim 20, wherein Z is —C(═O)R_{F}.

22. The compound of claim 20, wherein Z is —R_{F}.

23. The compound of claim 20, wherein R_{F} is fluoroalkyl.

24. The compound of claim 20, wherein R_{F} is perfluoroalkyl.

25. The compound of claim 20, wherein R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃.

26. The compound of claim 20, wherein W¹, W², W³, W⁴ and W⁵ are each —OH.

27. The compound of claim 20, wherein W¹, W², W³, W⁴ and W⁵ are each —OC(═O)CH₃.

28. The compound of claim 20, wherein M is —H.

29. The compound of claim 20, wherein M is —CH₃.

30. The compound of claim 20, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —H.

31. The compound of claim 20, wherein Z is —C(═O)R_{F}; R_{F} is —CF₃, —CH₂CF₃, —CF₂CF₃, —CH₂CH₂CF₃, —CH₂CH₂CH₂CF₃, —CH₂CH₂CF₂CF₃, —CH₂CH₂CF₂CF₂CF₃, or —CH₂CH₂(CF₂)₇CF₃; W¹, W², W³, W⁴ and W⁵ are each —OH; and M is —CH₃.

32. A compound represented by formula IV: 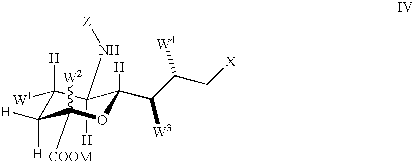
wherein
Z is —R_{F} or —C(═O)R³;
W¹, W², W³, and W⁴ are selected independently from the group consisting of —OR² and —OC(═O)R³;
M is hydrogen, alkyl, cycloalkyl, heterocycloalkyl, alkenyl, alkynyl, aryl, heteroaryl, aralkyl or heteroaralkyl;
R² and R³ are selected independently for each occurrence from the group consisting of hydrogen, alkyl, cycloalkyl, heterocycloalkyl, alkenyl, alkynyl, aryl, heteroaryl, aralkyl and heteroaralkyl; or two R² and R³ on being taken together are —(CH₂)ₙ—;
n is independently for each occurrence 1, 2, 3, 4 or 5;
X is selected from the group consisting of —R_{F}, —OR_{F}, —OC(═O)R_{F}, —NHR_{F}, —NHC(═O)R_{F}, —SR_{F} and —SC(═O)R_{F}; and
R_{F} is selected from the group consisting of fluoroalkyl, fluorocycloalkyl, fluoroheterocycloalkyl, fluoroalkenyl, fluoroalkynyl, fluoroaryl, fluoroheteroaryl, fluoroaralkyl, fluoroheteroaralkyl, fluoroacyl and fluorocarbonyl or a pharmaceutically acceptable salt thereof.

33. The compound of claim 32, wherein Z is —C(═O)R³.

34. The compound of claim 32, wherein Z is —R_{F}.

35. The compound of claim 32, wherein W¹, W², W³, and W⁴ are each —OH.

36. The compound of claim 32, wherein W¹, W², W³, and W⁴ are each —C(═O)CH₃.

37. The compound of claim 32, wherein M is —H.

38. The compound of claim 32, wherein M is —CH₃.

39. The compound of claim 32, wherein M is —CH₂CH₃.

40. The compound of claim 32, wherein R² and R³ are each alkyl.

41. The compound of claim 32, wherein R² and R³ are each —CH₃.

42. The compound of claim 32, wherein R_{F} is fluoroalkyl, fluoroaryl, or fluoroaralkyl.

43. The compound of claim 32, wherein X is —NHR_{F}, —NHC(═O)R_{F}, or —SR_{F}.

44. The compound of claim 32, wherein R_{F} is fluoroalkyl, fluoroaryl, or fluoroaralkyl; and X is —NHR_{F}, —NHC(═O)R_{F}, or —SR_{F}.

45. The compound of claim 32, wherein X is —S(CH₂)₂CF₃, —S(CH₂)₃CF₃, —S(CH—₂)₄CF₃, —SCH₂CF₂CF₃, —SCH₂(CF₂)₂CF₃, —SCH₂(CF₂)₃CF₃, —SCH₂(CF₂)₄CF₃, —NHCH—₂C₇F₁₅, —NHC(═O)(CH₂)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, or —NHC(═O)CH₂C₆F₅.

46. The compound of claim 32, wherein Z is —CH₂C₇H₁₅.

47. The compound of claim 32, wherein Z is —CH₂C₇H₁₅; X is —OC(═O)CH₃; W¹, W², W³, and W⁴ are each —C(═O)CH₃; and M is —CH₂CH₃.

48. The compound of claim 32, wherein Z is —C(═O)CH₃; W¹, W², W³, and W⁴ are each —OC(═O)CH₃; M is —CH₃; and X is —S(CH₂)₂CF₃, —S(CH₂)₃CF₃, —S(CH₂)₄CF₃, —SCH₂CF₂CF₃, —SCH₂(CF₂)₂CF₃, —SCH₂(CF₂)₃CF₃, —SCH₂(CF₂)₄CF₃, —NHC(═O)(CH₂—)₂CF₃, —NHC(═O)CH₂CF₂CF₃, —NHC(═O)CH₂(CF₂)₂CF₃, —NHC(═O)(CH₂)₂(CF₂)₂CF₃, —NHC(═O)CH₂(CF₂)₃CF₃, —NHC(═O)CH₂(CF₂)₇CF₃, or —NHC(═O)CH₂C₆F₅.

49. The compound of claim 32, wherein Z is —C(═O)CH₃; W¹, W², W³, and W⁴ are each —OC(═O)CH₃; M is —CH₂CH₃; and X is —NHCH₂C₇F₁₅.

50-98. (canceled)

99. A method of reducing cellular adhesion, comprising the steps of:
contacting said cell with a fluorine-containing monosaccharide; and
incubating said cell under conditions whereby the cell internalizes said fluorine-containing monosaccharide and extracellularly expresses a glucoconjugate comprising said fluorine-containing monosaccharide, or a derivative thereof, on the surface of said cell.

100-101. (canceled)

102. A method of imaging all or part of an organ of a mammal, comprising the steps of
administering to said mammal a detectable amount of a fluorine-containing monosaccharide or composition thereof; and
subjecting said mammal to nuclear magnetic resonance imaging.

103-108. (canceled)

109. A method of treating inflammation, comprising administering to a mammal in need thereof a therapeutically effective amount of a compound of claim 1; or a therapeutically effective amount of a pharmaceutical composition of claim 50.

110-116. (canceled)

117. A method of treating cancer, comprising administering to a mammal in need thereof a therapeutically effective amount of a compound of claim 1; or a therapeutically effective amount of a pharmaceutical composition of claim 50.

118-126. (canceled)
